(19) United States
(12) Patent Application Publication
(10) Pub. No.: US 2006/0014812 A1
(43) Pub. Date: Jan. 19, 2006

(54) Use of estrogen related receptor-modulating aryl ethers

(76) Inventors: Mark R. Player, Phoenixville, PA (US); Richard S. Pottorf, Belle Mead, NJ (US); Dionisios Rentzeperis, Downingtown, PA (US); Dibyendu De, Suwanee, GA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/177,716

(22) Filed: Jul. 8, 2005

Related U.S. Application Data
(60) Provisional application No. 60/587,850, filed on Jul. 14, 2004.

(51) Int. Cl. A61K 31/426; A61K 31/42; A61K 31/415; A61K 31/198; A61K 31/277

(52) U.S. Cl. 514/369; 514/378; 514/649; 514/621; 514/521; 514/567; 514/406

(57) ABSTRACT

Therapeutic methods of using certain heterocyclic arylidene aryl ether compounds for treating diseases or disorders mediated through modulation of estrogen related receptor alpha are described.

### CROSS-REFERENCE TO RELATED APPLICATION

This application claims priority to U.S. Provisional Application No. 60/587,850, filed Jul. 14, 2004.

### FIELD OF THE INVENTION

The present invention relates to methods of using certain heterocyclic arylidene aryl ether compounds for the treatment of disease states, disorders, and conditions mediated by estrogen related receptor alpha (ERR-α) activity.

### BACKGROUND OF THE INVENTION

Nuclear receptors are members of a superfamily of transcription factors. The members of this family share structural similarities and regulate a diverse set of biological effects (Olefsky, J. M. J. Biol. Chem. 2001, 276(40), 36863-36864). Ligands activate or repress these transcription factors that control genes involved in metabolism, differentiation and reproduction (Laudet, V. and H. Gronmeyer. The Nuclear Receptor Factbooks. 2002, San Diego: Academic Press). Presently, the human genome project has identified about 48 members for this family and cognate ligands have been identified for about 28 of them (Giguere, V. Endocrine Rev. 1999, 20(5), 689-725). This protein family is composed of modular structural domains that can be interchanged within the members of the family without loss of function. A typical nuclear receptor contains a hypervariable N-terminus, a conserved DNA binding domain (DBD), a hinge region, and a conserved ligand-binding domain (LBD). The function of the DBD is targeting of the receptor to specific DNA sequences (NHR response elements or NREs), and the function of the LBD is recognition of its cognate ligand. Within the sequence of the nuclear receptor there are regions involved in transcriptional activation. The AF-1 domain is situated at the N-terminus and constitutively activates transcription (Rochette-Egly, C. et al. Cell 1997, 90, 97-107; Rochette-Egly, C. et al. Mol. Endocrinol. 1992, 6, 2197-2209), while the AF-2 domain is embedded within the LBD and its transcriptional activation is ligand dependent (Wurtz, J. M. et al. Nat. Struct. Biol. 1996, 3, 87-94). Nuclear receptors can exist as monomers, homodimers or heterodimers and bind to direct or inverted nucleotide repeats (Laudet and Gronmeyer, 2002; Aranda, A. and A. Pascual. Physiol. Rev. 2001, 81(3), 1269-1304).

The members of this family exist either in an activated or repressed basal biological state. The basic mechanism of gene activation involves ligand dependent exchange of co-regulatory proteins. These co-regulatory proteins are referred to as co-activators or co-repressors (McKenna, L. J. et al. Endocrine Rev. 1999, 20, 321-344). A nuclear receptor in the repressed state is bound to its DNA response element and is associated with co-repressor proteins that recruit histone de-acetylases (HDACs) (Jones, P. L. and Y. B. Shi. Curr. Top. Microbiol. Immunol. 2003, 274, 237-268). In the presence of an agonist there is an exchange of co-repressors with co-activators that in turn recruit transcription factors that assemble into an ATP dependent chromatin-remodeling complex. Histones are hyper-acetylated, causing the nucleosome to unfold, and repression is alleviated. The AF-2 domain acts as the ligand dependent molecular switch for the exchange of co-regulatory proteins. In the presence of an agonist the AF-2 domain undergoes a conformational transition and presents a surface on the LBD for interaction with co-activator proteins. In the absence of an agonist or in the presence of an antagonist the AF-2 domain presents a surface that promotes interactions with co-repressor proteins. The interaction surfaces on the LBD for both co-activators, and co-repressors overlap and provide a conserved molecular mechanism for gene activation or repression that is shared by the members of this family of transcription factors (Xu, H. E. et al. Nature 2002, 415 (6873), 813-817).

Natural ligands that modulate the biological activity of nuclear receptors have been identified for only approximately one half of known nuclear receptors. Receptors for which no natural ligand has been identified are termed “orphan receptors” (Giguere, V., 1999). The discovery of ligands or compounds that interact with an orphan receptor will accelerate the understanding of the role of the nuclear receptors in physiology and disease and facilitate the pursuit of new therapeutic approaches. A sub-class of these receptors where no natural ligand has been identified is for the estrogen related receptors (ERRs).

ERR-α, an orphan receptor, is the first of the three identified members of the estrogen receptor related subfamily of orphan nuclear receptors (ERR-α, β, γ). The ERR subfamily is closely related to the estrogen receptors (ER-α and ER-β). ERR-α and ERR-β were first isolated by a low stringency hybridization screen (Giguere, V. et al. Nature 1988, 331, 91-94) followed later with the discovery of ERR-γ (Hong, H. et al. J. Biol. Chem. 1999, 274, 22618-22626). The ERRs and ERs share sequence similarity with the highest homology observed in their DBDs, approximately 60%, and all interact with the classical DNA estrogen response element. Recent biochemical evidence suggested that the ERRs and ERs share target genes, including pS2, lactoferin, aromatase and osteopontin, and share co-regulator proteins (Giguere, V. Trends in Endocrinol. Metab. 2002, 13, 220-225; Vanacker, J. M. et al. EMBO J. 1999,18, 4270-4279; Kraus, R. J. et al. J. Biol. Chem. 2002, 272, 24286-24834; Hong et al., 1999; Zhang, Z. and C. T. Teng. J. Biol. Chem. 2000, 275, 20387-20846). Therefore, one of the main functions of ERR is to regulate the response of estrogen responsive genes. The effect of the steroid hormone estrogen is primarily mediated in the breast, bone and endometrium. Thus, the identification of compounds that will interact with ERRs should provide a benefit for the treatment of bone related disease, breast cancer and reproduction.

ERR-α is shown to be present both in normal and breast cancer tissue (Ariazi, E. A. et al. Cancer Res. 2002, 62, 6510-6518). It has been reported that the main function of ERR-α in normal breast tissue is that of a repressor for estrogen responsive genes (Kraus et al., 2002). In breast cancers or cell lines that are non-estrogen responsive (ER-α negative), ERR-α has been reported to be in an activated state (Ariazi et al., 2002). Therefore, compounds that will interact with ERR-α may be useful agents for the treatment of breast cancer that is ER-α negative and non-responsive to classical anti-estrogenic therapy, or may be used as an adjunct agent for anti-estrogen responsive breast cancers. These agents may act as antagonists by reducing the biological activity of ERR-α in these particular tissues.

Many post-menopausal women experience osteoporosis, a condition that is a result of the reduction of estrogen production. Reduction of estrogen levels results in an increase of bone loss (Turner, R. T. et al. Endocrine Rev. 1994,15(3), 275-300). An anabolic effect on bone development has been observed on the administration of estrogens to postmenopausal patients with osteoporosis (Pacifici, R. J. Bone Miner. Res. 1996, 11(8), 1043-1051) but the molecular mechanism is unknown since ER-α and ER-β knock-out animals have minor skeletal defects, where the action of estrogens is typically mediated (Korach, K. S. Science 1994, 266, 1524-1527; Windahl, S. H. et al. J. Clin. Invest. 1999, 104(7), 895-901). Expression of ERR-α in bone is regulated by estrogen (Bonnelye, E. et al. Mol. Endocrin. 1997,11, 905-916; Bonnelye, E. et al. J. Cell Biol. 2001,153, 971-984). ERR-α is maintained throughout osteoblast differentiation stages. Overexpression of ERR-α in rat calvaria osteoblasts, an accepted model of bone differentiation, results in an increase of bone nodule formation, while treatment of rat calvaria osteoblasts with ERR-α antisense results in a decrease of bone nodule formation. ERR-α also regulates osteopontin, a protein believed to be involved in bone matrix formation (Bonnelye et al., 2001). Therefore compounds that will modulate ERR-α by increasing its activity may have an anabolic effect for the regeneration of bone density and provide a benefit over current approaches that prevent bone loss, but have no anabolic effect. Such compounds may enhance the activity of the receptor by two possible mechanisms: i) enhancing the association of the receptor with proteins that enhance its activity or improve the stability of the receptor; and ii) increasing the intracellular concentrations of the receptor and consequently increasing its activity. Conversely, with respect to bone diseases that are a result of abnormal bone growth, compounds that will interact with ERR-α and decrease its biological activity may provide a benefit for the treatment of these diseases by retarding bone growth. Antagonism of the association of the receptor with co-activator proteins decreases the activity of the receptor.

ERR-α is also present in cardiac, adipose, and muscle tissue and forms a transcriptional active complex with the PGC-1 co-activator family, co-activators implicated with energy homeostasis, mitochondria biogenesis, hepatic gluconeogenesis and in the regulation of genes involved in fatty acid beta-oxidation (Kamei, Y. et al. Proc. Natl. Acad. Sci. USA 2003, 100(21), 12378-12383). ERR-α regulates the expression of the medium chain acyl-CoA dehydrogenase promoter (MCAD). Medium chain acyl-CoA dehydrogenase is a gene involved in the initial reaction in fatty acid beta-oxidation. It is believed that in the adipose tissue ERR-α regulates energy expenditure through the regulation of MCAD (Sladek, R. et al. Mol. Cell. Biol. 1997, 17, 5400-5409; Vega, R. B. and D. P. Kelly. J. Biol. Chem. 1997, 272, 31693-31699). In antisense experiments in rat calvaria osteoblasts, in addition to the inhibition of bone nodule formation, there was an increase in adipocyte differentiation markers including aP2 and PPAR-γ (Bonnelye, E. et al. Endocrinology 2002,143, 3658-3670). Recently an ERR-α knockout model has been described that exhibited reduced fat mass relative to the wild type and DNA chip analysis data indicated alteration of the expression levels of genes involved in adipogenesis and energy metabolism (Luo, J. et al. Mol. Cell. Biol. 2003, 23(22), 7947-7956). More recently it has been shown that ERR-α regulates the expression of endothelial nitric oxide synthase, a gene that has a protective mechanism against arteriosclerosis (Sumi, D. and L. J. Ignarro. Proc Natl. Acad. Sci. 2003, 100, 14451-14456). The biochemical evidence supports the involvement of ERR-α in metabolic homeostasis and differentiation of cells into adipocytes. Therefore, compounds interacting with ERR-α can affect energy homeostasis and may therefore provide a benefit for the treatment of obesity and metabolic syndrome related disease indications, including arteriosclerosis and diabetes (Grundy, S. M. et al. Circulation 2004,109(3), 433-438).

X-Ceptor Therapeutics (San Diego, Calif.) has indicated that it has preclinical ERR-α antagonists, although their structures were not reported (The Knowledge Foundation 3^{rd} Annual Orphan & Nuclear Receptors Meeting, San Diego, Calif. October 2003). Lion Bioscience AG has disclosed the use of certain pyrazole derivatives as antagonists of ERR-α for treating cancer, osteoporosis, obesity, lipid disorders and cardiovascular disorders and for regulating fertility (European Published Patent Application 1398029).

### SUMMARY OF THE INVENTION

The invention relates to methods of treating a subject suffering from or diagnosed with a disease, disorder, or medical condition mediated by ERR-α activity, comprising administering to the subject an effective amount to treat the disease, disorder, or medical condition of a compound of formula (I): 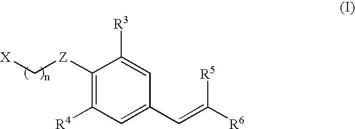
wherein:
n is 0 or 1;
Z is —O—, —S—, >NH, or >NR^{a} where R^{a} is alkyl, cycloalkyl, phenyl, or heterocycloalkyl; X is an aryl or heteroaryl group;
R³ is —H or —O-alkyl unsubstituted or substituted with one or more substituents independently selected from the group consisting of —OH, halo, —CN, —O-alkyl, and —N(R^{w})R^{x} where R^{w} and R^{x} are each independently —H or alkyl;
R⁴ is selected from the group consisting of —H, halo, —O-alkyl, —CN, —NO₂, and —COOH; and
R and R are each independently —CN; —COOH; or a moiety selected from the group consisting of —COO-alkyl, —(C═O)alkyl, —(S═(O)ₘ)-aryl where m is 0, 1, or 2, cycloalkyl, heterocycloalkyl, —(C═O)phenyl, heteroaryl, and —(C═O)heterocycloalkyl; or R⁵ and R⁶ taken together with the carbon to which they are attached form an optionally benzofused heterocycloalkyl or cycloalkyl moiety; wherein each such moiety is unsubstituted or substituted with one or more substituents independently selected from the group consisting of: —OH; ═O; ═S; alkyl optionally substituted with —OH, —O-alkyl, phenyl, —NH₂, —NH(alkyl), —N(alkyl)₂, halo, —CF₃, —COOH, or —COO-alkyl; —O-alkyl; phenyl; —O-phenyl;

benzyl; —O-benzyl; cycloalkyl; —O-cycloalkyl; —CN; —NO₂; —N(R^{y})R^{z} where R^{y} and R^{z} are each independently —H, alkyl, or —(C═O)alkyl, or R^{y} and R^{z} taken together with the nitrogen to which they are attached form a heterocycloalkyl wherein one carbon ring atom is optionally replaced with >O, >NH or >N-alkyl and where one carbon ring atom is optionally substituted with —OH or ═O; —(C═O)N(R^{y})R^{z}; —(N—R^{t})SO₂alkyl where R^{t} is —H or alkyl; -(C═O)alkyl; —(S═(O)n)alkyl where n is 0, 1 or 2; —SO₂N(R^{y})R^{z} where R^{y} and R^{z} are as defined above; —SCF₃; halo; —CF₃; —OCF₃; —COOH; and —COOalkyl.;

or a pharmaceutically acceptable salt, pharmaceutically acceptable prodrug, or pharmaceutically active metabolite of such compound.

In preferred embodiments, pharmaceutical agents of the present invention are used to treat bone-related disease, bone formation, cartilage formation, cartilage loss, cartilage degeneration, cartilage injury, ankylosing spondylitis, chronic back injury, gout, osteoporosis, osteolytic bone metastasis (for example, from breast cancer), multiple myeloma, chondrosarcoma, chondrodysplasia, osteogenesis imperfecta, osteomalacia, Paget's disease, polymyalgia rheumatica, pseudogout, arthritis, rheumatoid arthritis, infectious arthritis, osteoarthritis, psoriatic arthritis, reactive arthritis, childhood arthritis, Reiter's syndrome, or repetitive stress injury.

In other preferred embodiments, the agents are used for treating periodontal disease, chronic inflammatory airway disease, chronic bronchitis, or chronic obstructive pulmonary disease.

In additional preferred embodiments, the agents are used for treating breast cancer, such as breast cancer unresponsive to anti-estrogen therapy.

In other preferred embodiments, agents of the present invention are used for treating metabolic syndrome, obesity, disorders of energy homeostasis, diabetes, lipid disorders, cardiovascular disorders, or artherosclerosis.

Additional aspects, embodiments, features, and advantages of the invention will be apparent from the following detailed description and appended claims.

### DETAILED DESCRIPTION AND PREFERRED EMBODIMENTS

The invention as defined in the claims will be more fully appreciated by reference to the following description, including the terms defined below.

Preferred compounds used in the methods of the invention include compounds represented by Formula (I) as defined above.

Preferably, X is an aryl or heteroaryl group having one ring or two fused rings, wherein each ring has five or six ring atoms.

In preferred embodiments, subjects are treated by administering compounds of Formula (I) wherein:
n is 0 or 1 as defined above; and
Z is —O—;
X is 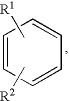
   where
R¹ and R² are each independently —H; halo; —CN; —CF₃; —NO₂; —COOH; or a moiety selected from the group consisting of: —C₁₋₆alkyl, —OC₁₋₆alkyl, —C₂₋₆alkenyl, —OC₃₋₆alkenyl, —C₂₋₆alkynyl, —OC₃₋₆alkynyl, —C₃₋₇cycloalkyl, —(C₃₋₈cycloalkyl)C₁₋₆alkyl, —(C₃₋₈cycloalkyl)C₃₋₈alkenyl, —C₀₋₈alkylC(═O)C₁₋₈alkyl, 5-9 membered heterocycloalkyl, phenyl, —O-phenyl, benzyl, -(5-9-membered heterocycloalkyl)C₁₋₆alkyl, -(phenyl)C₁₋₆alkyl, —COOC₁₋₆alkyl, and —(C═O)N(R^{s})R^{t} where R^{s} and R^{t} are each independently —H or —C₁₋₆alkyl; wherein each said moiety is unsubstituted or substituted with one or more substituents independently selected from the group consisting of —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, and —N(R^{w})R^{x} where R^{w} and R^{x} are each independently —H or —C₁₋₆alkyl;
R⁴ is selected from the group consisting of —H, halo, —OC₁₋₆alkyl, —CN, —NO₂, and —COOH; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, and —(C═O)(5-9 membered heterocycloalkyl); or R⁵ and R⁶ taken together with the carbon to which they are attached form an optionally benzofused 5-9 membered heterocycloalkyl or cycloalkyl moiety; wherein each said moiety is unsubstituted or substituted with one or more substituents independently selected from the group consisting of: —OH; ═O; ═S; —C₁₋₆alkyl optionally substituted with —OH, —OC₁₋₆alkyl, phenyl, —NH₂, —NH(C₁₋₆alkyl), —N(C₁₋₆alkyl)₂, halo, —CF₃, —COOH, or —COOC₁₋₆alkyl; —OC₁₋₆alkyl; phenyl; —Ophenyl; benzyl; —Obenzyl; —C₃₋₆cycloalkyl; —OC₃₋₆cycloalkyl; —CN; —NO₂; —N(R^{y})R^{z} where R^{y} and R^{z} are each independently —H, —C₁₋₆alkyl, or —(C═O)C₁₋₆alkyl, or R^{y} and R^{z} taken together with the nitrogen to which they are attached form a 4-7 membered heterocycloalkyl ring wherein one carbon ring atom is optionally replaced with >O, >NH or >N(C₁₋₄alkyl) and where one carbon ring atom is optionally substituted with —OH or ═O; —(C═O)N(R^{y})R^{z}; —(N—R^{t})SO₂C₁₋₆alkyl where R^{t} is —H or —C₁₋₆alkyl; —(C═O)C₁₋₆alkyl; —(S═(O)ₙ)—C₁₋₆alkyl where n is 0, 1 or 2; —SO₂N(R^{y})R^{z} where R^{y} and R^{z} are as defined above; —SCF₃; halo; —CF₃; —OCF₃; —COOH; and —COOC₁₋₆alkyl.

In further preferred embodiments, subjects are treated by administering compounds represented by the Formula (II): 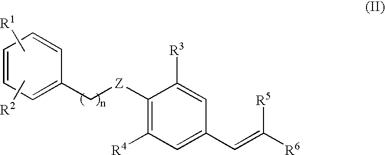
where R¹, R², n, Z, R³, R⁴, R⁵, and R⁶ are as herein defined.

In still further preferred embodiments, subjects are treated by administering compounds of Formula (II) wherein:
n is 0 or 1;
Z is —O—, —S—, >NH, or >NR^{a} where R^{a} is alkyl, —C₁₋₆cycloalkyl, phenyl, or 5-9-membered heterocycloalkyl;
R¹ and R² are each independently —H, halo, —CN, —CF₃, —NO₂, or —COOH, or a moiety selected from the group consisting of: —C₁₋₆alkyl, —OC₁₋₆alkyl, —C₂₋₆alkenyl, —OC₃₋₆alkenyl, —C₂₋₆alkynyl, —OC₃₋₆alkynyl, —C₃₋₇cycloalkyl, —(C₃₋₈cycloalkyl)C₁₋₆alkyl, —(C₃₋₈cycloalkyl)—C₃₋₈alkenyl, —C₀₋₈alkylC(═O)C₁₋₈alkyl, 5-9 membered heterocycloalkyl, phenyl, —O-phenyl, benzyl, -(5-9-membered heterocycloalkyl)C₁₋₆alkylene, -(phenyl)C₁₋₆alkyl, —COOC₁₋₆alkyl, and —(C═O)N(R^{s})R^{t} where R^{s} and R^{t} are each independently —H or —C₁₋₆alkyl; wherein each said moiety is unsubstituted or substituted with one or more substituents independently selected from the group consisting of —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, and —NR^{w}R^{x} where R^{w} and R^{x} are each independently —H or —C₁₋₆alkyl;
R⁴ is —H, —OCH₃, or —Cl; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, and —(C═O)(5-9 membered heterocycloalkyl); or R⁵ and R⁶ taken together with the carbon to which they are attached form a 5-9 membered heterocycloalkyl or cycloalkyl moiety; wherein each said moiety is unsubstituted or substituted with one or more substituents independently selected from the group consisting of: —OH; —C₁₋₆alkyl; —OC₁₋₆alkyl; —Ophenyl; benzyl; —Obenzyl; —C₃₋₆cycloalkyl; —OC₃₋₆cycloalkyl; —CN; —NO₂; —N(R^{y})R^{z} where R^{y} and R^{z} are each independently —H, —C₁₋₆alkyl, —C₁₋₆alkenyl, or —(C═O)C₁₋₆alkyl, or R^{y} and R^{z} taken together with the nitrogen to which they are attached form a 4-7 membered heterocycloalkyl ring wherein one carbon ring atom is optionally replaced with >O, ═N—, >NH or >N(C₁₋₄alkyl) and where one carbon ring atom is optionally substituted with —OH or ═O; —(C═O)N(R^{y})R^{z}; —(N—R^{t})SO₂C₁₋₆alkyl where R^{t} is —H or —C₁₋₆alkyl; —(C═O)C₁₋₆alkyl; —(S═(O)ₙ)—C₁₋₆alkyl where n is 0, 1 or 2; —SO₂N(R^{y})R^{z} where R^{y} and R^{z} are as defined above; —SCF₃; halo; —CF₃; —OCF₃; —COOH; and —COOC₁₋₆alkyl; and pharmaceutically acceptable salts, pharmaceutically acceptable prodrugs, and pharmaceutically active metabolites of said compounds.

In still further preferred embodiments, subjects are treated by administering compounds of Formula (II) wherein:
n is 0 or 1;
Z is —O—;
R¹ and R² are each independently —H, -halo, —CN, —CF₃, —NO₂, or —COOH, or a moiety selected from the group consisting of: —C₁₋₆alkyl, —OC₁₋₆alkyl, —C₃₋₇cycloalkyl, —(C═O)C₁₋₆alkyl, —COOC₁₋₆alkyl, —(C═O)N(R^{s})R^{t} where R^{s} and R^{t} are each independently —H or —C₁₋₆alkyl, wherein each said moiety is unsubstituted or substituted with one or more substituents independently selected from the group consisting of: —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, or —NR^{w}R^{x} where R^{w} and R^{x} are each independently —H or —C₁₋₆alkyl;
R⁴ is —H or —Cl; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, —(C═O)(5-9 membered heterocycloalkyl); or R⁵ and R⁶ taken together with the carbon to which they are attached form a 5-9 membered heterocycloalkyl or cycloalkyl moiety selected from the group consisting of: 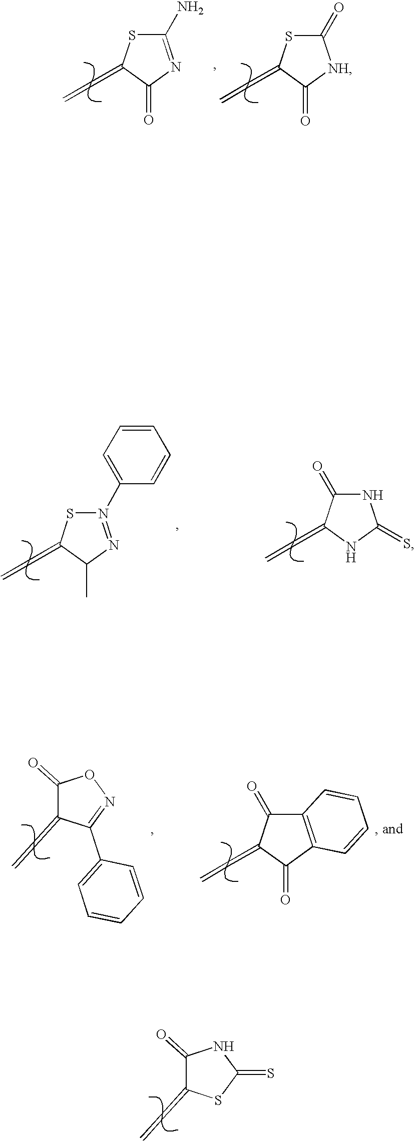
   wherein each said moiety is unsubstituted or substituted with one or more substituents independently selected from the group consisting of: —OH; —C₁₋₄alkyl; —OC₁₋₃alkyl; phenyl; benzyl; —C₃₋₆cycloalkyl; —OC₃₋₆cycloalkyl; —CN; —NO₂; —N(R^{y})R^{z} where R^{y} and R^{z} are each independently —H or —C₁₋₆alkyl, or where R^{y} and R^{z} may be taken together with the nitrogen to which they are attached to form a 4-7 membered heterocycloalkyl ring wherein one carbon ring atom is optionally replaced with >O, ═N—, >NH or >N(C₁₋₄alkyl) and where one carbon ring atom is optionally substituted with —OH; —(C═O)N(R^{y})R^{z}; —(N—R^{t})SO₂C₁₋₆alkyl where R^{t} is —H or —C₁₋₆alkyl; —(C═O)C₁₋₆alkyl; —(S═(O)ₙ)—C₁₋₆alkyl where n is 0, 1 or 2; —SO₂N(R^{y})R^{z}; -halo; —CF₃; —OCF₃; —COOH; and —COOC₁₋₆alkyl.

In still further preferred embodiments, subjects are treated by administering compounds of Formula (II) wherein:
n is 0 or 1;
Z is —O—;
R¹ and R² are each independently selected from the group consisting of: —H, —OCH₃, —F, —Cl, —CN, —CF₃, —NO₂, and —COOCH₃;
R³ is —H or —OCH₃;
R⁴ is —H or —Cl; and
R⁵ and R⁶ are each independently —CN; or a moiety selected from the group consisting of —COOC₁₋₆alkyl, —(C═O)phenyl, and 3-pyrazolyl; or R⁵ and R⁶ taken together with the carbon to which they are attached form a 5-9 membered heterocyclic or carbocyclic moiety selected from the group consisting of: 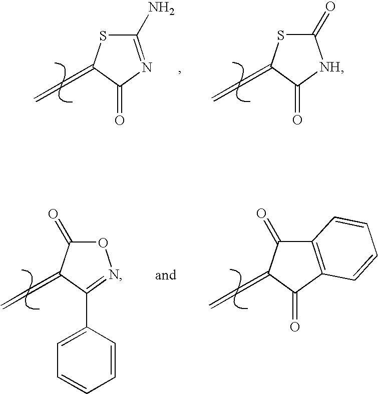
   each unsubstituted or substituted with one or more substituents selected from the group consisting of: —OH, —C₁₋₄alkyl, —OC₁₋₃alkyl, phenyl, —C₃₋₆cycloalkyl, —OC₃₋₆cycloalkyl, —CN, —NO₂, —NH₂, —N(C₁₋₃alkyl)₂, —N-piperidinyl, —N-morpholinyl, —N-thiomorpholinyl, —(C═O)N(C₁₋₃alkyl)₂, —(N—R^{t})SO₂C₁₋₃alkyl where R^{t} is —H or —C₁₋₆alkyl, —(C═O)C₁₋₃alkyl, —(S═(O)ₙ)—C₁₋₃alkyl where n is 0, 1 or 2, —SO₂N(C₁₋₃alkyl)₂, -halo, —CF₃, —OCF₃, —COOH, and —COOC₁₋₆alkyl.

In still further preferred embodiments, subjects are treated by administering a compound or compounds selected from the group consisting of:
5-Amino-3-{1-cyano-2-[3-methoxy-4-(2-nitro-4-trifluoromethyl-phenoxy)-phenyl]vinyl}-1-phenyl-1H-pyrazole-4-carbonitrile;
5-Amino-3-{1-cyano-2-[4-(2-nitro-4-trifluoromethyl-phenoxy)-phenyl]-vinyl}-1-phenyl-1H-pyrazole-4-carbonitrile;
5-Amino-3-{1-cyano-2-[3-methoxy-4-(4-nitro-3-trifluoromethyl-phenoxy)-phenyl]-vinyl}-1-phenyl-1H-pyrazole-4-carbonitrile;
5-Amino-3-{1-cyano-2-[4-(4-cyano-3-trifluoromethyl-phenoxy)-3-methoxy-phenyl]-vinyl}-1-phenyl-1H-pyrazole-4-carbonitrile;
5-Amino-3-[1-cyano-2-(4-phenoxy-phenyl)-vinyl]-1-phenyl-1H-pyrazole-4-carbonitrile;
5-Amino-3-[2-(4-benzyloxy-3-methoxy-phenyl)-1-cyano-vinyl]-1-phenyl-1H-pyrazole-4-carbonitrile;
2-Amino-5-[3-methoxy-4-(2-nitro-4-trifluoromethyl-phenoxy)-benzylidene]-thiazol-4-one;
4-[4-(2-Amino-4-oxo-4H-thiazol-5-ylidenemethyl)-2-methoxy-phenoxy]-2-trifluoromethyl-benzonitrile;
2-Amino-5-[4-(4-methoxy-phenoxy)-benzylidene]-thiazol-4-one;
5-[3-Methoxy-4-(2-nitro-4-trifluoromethyl-phenoxy)-benzylidene]-thiazolidine-2,4-dione;
5-[3-Methoxy-4-(4-nitro-3-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzonitrile;
5-[3-Chloro-5-methoxy-4-(2-nitro-4-trifluoromethyl-phenoxy)-benzylidene]-thiazolidine-2,4-dione;
5-[4-(2-Nitro-4-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzoic acid methyl ester;
5-[4-(4-Methoxy-phenoxy)-benzylidene]-thiazolidine-2,4-dione;
{5-[3-Methoxy-4-(2-nitro-4-trifluoromethyl-phenoxy)-benzylidene]-2,4-dioxo-thiazolidin-3-yl}-acetic acid ethyl ester;
4-[4-(2,4-Dioxo-thiazolidin-5-ylidenemethyl)-2-methoxy-phenoxy]-3-nitro-benzoic acid methyl ester;
3-Butyl-5-[3-methoxy-4-(2-nitro-4-trifluoromethyl-phenoxy)-benzylidene]-thiazolidine-2,4-dione;
5-(4-Phenoxy-benzylidene)-thiazolidine-2,4-dione;
5-[3-(3-Chloro-phenoxy)-benzylidene]-thiazolidine-2,4-dione;
2-[3-Methoxy-4-(2-nitro-4-trifluoromethyl-phenoxy)-benzylidene]-malononitrile;
2-Benzenesulfonyl-3-[3-methoxy-4-(2-nitro-4-trifluoromethyl-phenoxy)-phenyl]-acrylonitrile;
2—Cyano-3-[3-methoxy-4-(2-nitro-4-trifluoromethyl-phenoxy)-phenyl]-acrylic acid ethyl ester;
3-[3-Methoxy-4-(2-nitro-4-trifluoromethyl-phenoxy)-phenyl]-2-thiophen-2-yl-acrylonitrile;
2-(1H-Benzoimidazol-2-yl)-3-[3-methoxy-4-(2-nitro-4-trifluoromethyl-phenoxy)-phenyl]-acrylonitrile;
3-[3-Methoxy-4-(2-nitro-4-trifluoromethyl-phenoxy)-phenyl]-2-pyridin-2-yl-acrylonitrile;
2-Benzoyl-3-[3-methoxy-4-(2-nitro-4-trifluoromethyl-phenoxy)-phenyl]-acrylonitrile;
4,4,4-Trifluoro-2-[3-methoxy-4-(2-nitro-4-trifluoromethyl-phenoxy)-benzylidene]-1-thiophen-2-yl-butane-1,3-dione;
4-[3-Methoxy-4-(2-nitro-4-trifluoromethyl-phenoxy)-benzylidene]-5-methyl-2-phenyl-2,4-dihydro-pyrazol-3-one;
5-[3-Methoxy-4-(2-nitro-4-trifluoromethyl-phenoxy)-benzylidene]-2-thioxo-imidazolidin-4-one;
4-[3-Methoxy-4-(2-nitro-4-trifluoromethyl-phenoxy)-benzylidene]-3-phenyl-4H-isoxazol-5-one;
2-[3-Methoxy-4-(2-nitro-4-trifluoromethyl-phenoxy)-benzylidene]-indan-1,3-dione.
5-[3-Methoxy-4-(2-nitro-4-trifluoromethyl-phenoxy)-benzylidene]-3-phenyl-2-thioxo-imidazolidin-4-one;
4-[4-(1,3-Dioxo-indan-2-ylidenemethyl)-2-methoxy-phenoxy]-3-nitro-benzoic acid methyl ester;
3-Ethyl-5-[3-methoxy-4-(2-nitro-4-trifluoromethyl-phenoxy)-benzylidene]-2-thioxo-thiazolidin-4-one;
5-[4-(2-Chloro-4-fluoro-benzyloxy)-3-methoxy-benzylidene]-thiazolidine-2,4-dione;
5-[4-(3-Fluoro-benzyloxy)-3-methoxy-benzylidene]-thiazolidine-2,4-dione;
5-(4-Benzyloxy-3-methoxy-benzylidene)-thiazolidine-2,4-dione; and
2-[4-(3-Fluoro-benzyloxy)-3-methoxy-benzylidene]-indan-1,3-dione.

In still further preferred embodiments, subjects are treated by administering a compound or compounds selected from the group consisting of 5-[4-(2-chloro-4-fluoro-benzyloxy)-3-methoxy-benzylidene]-thiazolidine-2,4-dione and 5-[4-(3-fluoro-benzyloxy)-3-methoxy-benzylidene]-thiazolidine-2,4-dione.

The pharmaceutical agents useful in the inventive method also include pharmaceutically acceptable salts, prodrugs, and active metabolites of compounds of Formula (I) or (II).

As used herein, the terms “including” and “comprising” are used herein in their open, non-limiting sense.

The term “alkyl” refers to a straight- or branched-chain alkyl group having from 1 to 12 carbon atoms in the chain. Exemplary alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and the like. The term “alkylene” refers to a divalent straight- or branched-chain alkyl group having from 1 to 12 carbon atoms in the chain. Exemplary alkylene groups include methylene, ethylene, propylene, and the like.

The term “alkenyl” refers to a straight- or branched-chain alkenyl group having from 2 to 12 carbon atoms in the chain. Illustrative alkenyl groups include prop-2-enyl, but-2-enyl, but-3-enyl, 2-methylprop-2-enyl, hex-2-enyl, and the like.

The term “alkynyl” refers to a straight- or branched-chain alkynyl group having from 2 to 12 carbon atoms in the chain. Illustrative alkynyl groups include prop-2-ynyl, but-2-ynyl, but-3-ynyl, 2-methylbut-2-ynyl, hex-2-ynyl, and the like.

The term “aryl” (Ar) refers to a monocyclic, or fused or spiro polycyclic, aromatic carbocycle (ring structure having ring atoms that are all carbon) having from 3 to 12 ring atoms per ring. Illustrative examples of aryl groups include the following moieties:

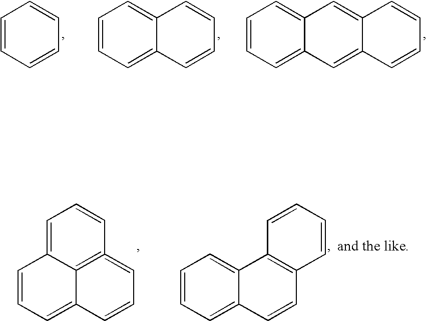

The term “heteroaryl” (heteroAr) refers to a monocyclic, or fused or spiro polycyclic, aromatic heterocycle (ring structure having ring atoms selected from carbon atoms as well as nitrogen, oxygen, and sulfur heteroatoms) having from 3 to 12 ring atoms per ring. Illustrative examples of aryl groups include the following moieties:

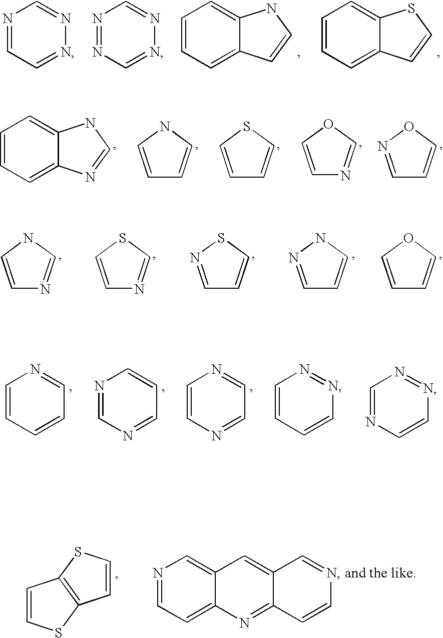

The term “cycloalkyl” refers to a saturated or partially saturated, monocyclic or fused or spiro polycyclic, carbocycle having from 3 to 12 ring atoms per ring. Illustrative examples of cycloalkyl groups include the following moieties:

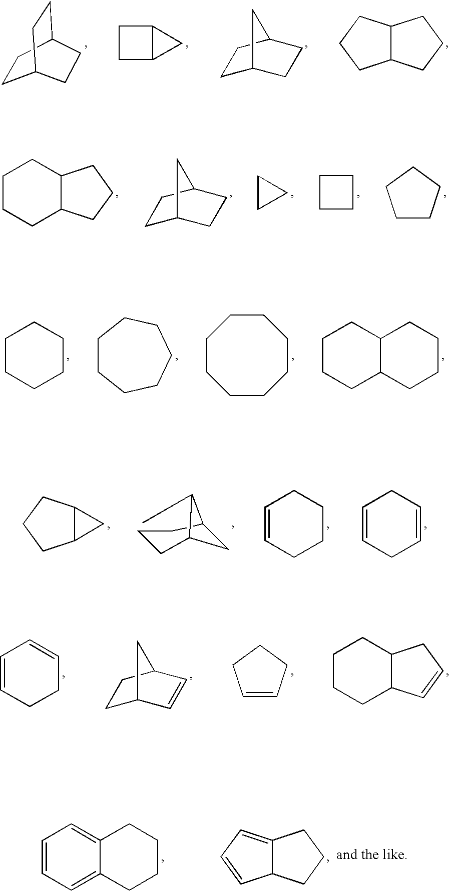

A “heterocycloalkyl” refers to a monocyclic, or fused or spiro polycyclic, ring structure that is saturated or partially saturated and has from 3 to 12 ring atoms per ring selected from C atoms and N, O, and S heteroatoms. Illustrative examples of heterocycloalkyl groups include:

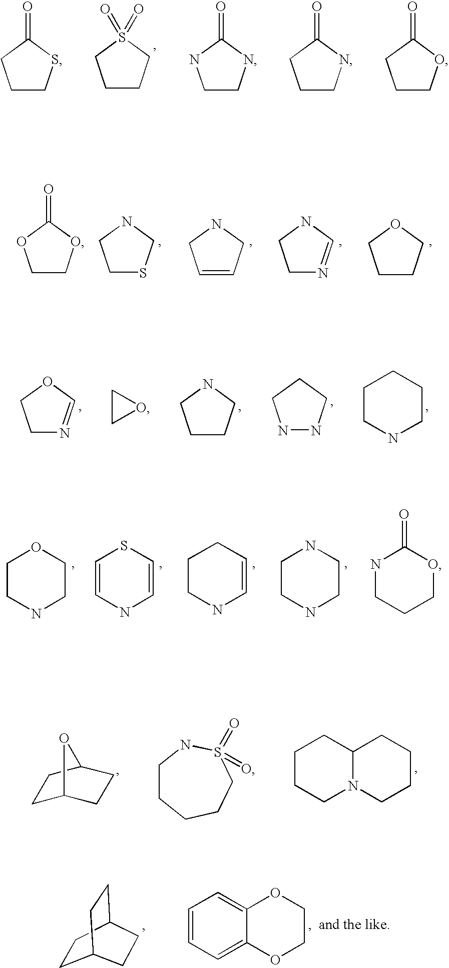

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents.

Each of Formula (I) and (II) is intended to represent compounds having structures depicted by the structural formula as well as certain variations or forms. In particular, compounds of Formula (I) or (II) may have asymmetric centers and therefore exist in different enantiomeric forms. All optical isomers and stereoisomers of the compounds of the formula, and mixtures thereof, are considered within the scope of the formula. With respect to the compounds of Formula (I) or (II), the invention includes the use of a racemate, one or more enantiomeric forms, one or more diastereomeric forms, or mixtures thereof.

Furthermore, certain structures depicted by Formula (I) or (II) may exist as geometric isomers (i.e., cis and trans isomers) or as tautomers. Additionally, Formula (I) is intended to represent hydrates, solvates, and polymorphs of such compounds, and mixtures thereof.

Formula (I) and (II) are each also intended to represent isotopically labeled forms of the compounds. Isotopically labeled compounds have structures depicted by Formula (I) or (II) except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²p, ³⁵S, ¹⁸F, and ³⁶Cl, respectively. Various isotopically-labelled compounds of the present invention, for example those into which radioactive isotopes such as ³H, ¹¹C, and ¹⁴C are incorporated, are useful in drug or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of Formula (I) of this invention and prodrugs thereof can generally be prepared by carrying out the procedures disclosed in the schemes or in the examples and preparations described below by substituting a readily available isotopically labeled reagent for a non-isotopically labeled reagent.

The invention includes also pharmaceutically acceptable salts of the compounds represented by Formula (I) or (II). A “pharmaceutically acceptable salt” is intended to mean a salt of a free acid or base of a compound represented by Formula (I) that is not toxic, biologically intolerable, or otherwise biologically undesirable. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Exemplary pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) or (II) is a base, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, phosphoric acid and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid or tartaric acid, an amino acid, such as aspartic acid or glutamic acid, an aromatic acid, such as benzoic acid, 2-acetoxybenzoic acid or cinnamic acid, a sulfonic acid, such as laurylsulfonic acid, p-toluenesulfonic acid, methanesulfonic acid or ethanesulfonic acid, or the like.

If the compound of Formula (I) or (II) is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine and piperazine, and inorganic salts derived from sodium, calcium, potassium, magnesium, manganese, iron, copper, zinc, aluminum and lithium.

The invention also relates to treatment methods employing pharmaceutically acceptable prodrugs of the compounds represented by Formula (I) and (II). The term “prodrug” means a precursor of a compound of the specified formula that, following administration to a subject, yields the compound in vivo via a chemical or physiological process such as solvolysis or physiological conditions (e.g., a prodrug on being brought to physiological pH is converted to the compound of Formula (I) or (II)). A “pharmaceutically acceptable prodrug” is a prodrug that is not toxic, biologically intolerable, or otherwise biologically unsuitable for administration to the subject.

Exemplary prodrugs include compounds having an amino acid residue, or a polypeptide chain of two or more (e.g., two, three or four) amino acid residues, covalently joined through an amide or ester bond to a free amino, hydroxy or carboxylic acid group of a compound of Formula (I) or (II). Examples of amino acid residues include the twenty naturally occurring amino acids commonly designated by three letter symbols as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) or (II) as amides or alkyl esters. Exemplary amides include those derived from ammonia, primary C₁₋₆alkyl amines and secondary di(C₁₋₆alkyl) amines. Secondary amines include 5- or 6-membered heterocycloalkyl or heteroaryl ring moieties having from 1 to 3 heteroatoms where at least one is a nitrogen atom. Preferred amides are derived from ammonia, C₁₋₃alkyl primary amines, and di(C₁₋₂alkyl)amines. Exemplary esters of the invention include C₁₋₇alkyl, C₅₋₇carbocyclyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Advanced Drug Delivery Reviews, 1996, 19, 115. Carbamate derivatives of hydroxy and amino groups also yield prodrugs. Carbonate derivatives, sulfonate esters and sulfate esters of hydroxy groups also provide prodrugs. Derivatization of hydroxy groups as (acyloxy)methyl and (acyloxy)ethyl ethers, wherein the acyl group may be an alkyl ester, optionally substituted with one or more ether, amine or carboxylic acid functionalities, or where the acyl group is an amino acid ester as described above, are also encompassed. Prodrugs of this type may be prepared as described in J. Med. Chem. 1996, 39, 10. Free amines can also be derivatized as amides, sulfonamides or phosphonamides. All of these prodrug moieties may incorporate groups including ether, amine and carboxylic acid functionalities.

Pharmaceutically active metabolites may also be used in the methods of the invention. A “pharmaceutically active metabolite” means a pharmacologically active product of metabolism in the body of a compound of Formula (I) or (II) or salt thereof. Prodrugs and active metabolites of a compound may be determined using routine techniques known in the art. See, e.g., Bertolini et al., J. Med. Chem. 1997, 40, 2011-2016; Shan et al., J. Pharm. Sci. 86 (7), 765-767; Bagshawe, Drug Dev. Res. 1995, 34, 220-230; Bodor, Advances in Drug Res. 1984, 13, 224-331; Bundgaard, Design of Prodrugs (Elsevier Press 1985); and Larsen, Design and Application of Prodrugs, Drug Design and Development (Krogsgaard-Larsen et al., eds., Harwood Academic Publishers, 1991).

The compounds represented by Formula (I) and (II) and their pharmaceutically acceptable salts, pharmaceutically acceptable prodrugs, and pharmaceutically active metabolites (collectively, “agents”) of the present invention are useful as estrogen related receptor alpha modulators in the methods of the invention. Preferred agents are ERR-α antagonists. The agents may be used in the inventive methods for the treatment or prevention of bone-related disease, breast cancer (e.g., diagnosed as unresponsive to estrogen therapy), and obesity.

Thus, the pharmaceutical agents are used to treat subjects diagnosed with or suffering from a disorder or condition mediated through ERR-α activity. Preferably, the treatment comprises administering to a subject an effective amount to treat the disorder or condition by increasing the activity of ERR-α through an increase of the stabilization of the receptor. The term “treat” or “treating” as used herein is intended to refer to administration of an agent or composition of the invention to a subject for the purpose of effecting a therapeutic or prophylactic benefit through modulation of ERR-α activity. Treating includes reversing, ameliorating, alleviating, inhibiting the progress of, lessening the severity of, or preventing a disorder or condition, or one or more symptoms of such disorder or condition. The term “subject” refers to a mammalian patient, such as a human.

Accordingly, the invention relates to methods of using the pharmaceutical agents to treat subjects diagnosed with or suffering from a disorder or condition mediated through ERR-α activity, such as: bone-related disease, bone formation, cartilage formation, cartilage loss, cartilage degeneration, cartilage injury, ankylosing spondylitis, chronic back injury, gout, osteoporosis, osteolytic bone metastasis (for example, from breast cancer), multiple myeloma, chondrosarcoma, chondrodysplasia, osteogenesis imperfecta, osteomalacia, Paget's disease, polymyalgia rheumatica, pseudogout, arthritis, rheumatoid arthritis, infectious arthritis, osteoarthritis, psoriatic arthritis, reactive arthritis, childhood arthritis, Reiter's syndrome, repetitive stress injury, periodontal disease, chronic inflammatory airway disease, chronic bronchitis, chronic obstructive pulmonary disease, breast cancer (e.g., breast cancer unresponsive to anti-estrogen therapy), metabolic syndrome, obesity, disorders of energy, homeostasis, diabetes, lipid disorders, cardiovascular disorders, or artherosclerosis.

In a treatment method according to the invention, an effective amount of a pharmaceutical agent according to the invention is administered to a patient suffering from or diagnosed as having such a disorder or condition. An “effective amount” means an amount or dose generally sufficient to bring about the desired therapeutic or prophylactic benefit in subjects undergoing treatment.

Effective amounts or doses of the agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disorder or condition, the subject's previous or ongoing therapy, the subject's health status and response to drugs, and the judgment of the treating physician. An exemplary dose is in the range of from about 0.001 to about 200 mg per kg of subject's body weight per day, preferably about 0.05 to 100 mg/kg/day, or about 1 to 35 mg/kg/day, in single or divided dosage units (e.g., BID, TID, QID). For a 70-kg human, an illustrative dosage amount is from about 0.05 to about 7 g/day, or about 0.2 to about 2.5 g/day.

Once improvement of the patient's conditions has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

The agents of the invention are used, alone or in combination with one or more other active ingredients, to formulate pharmaceutical compositions of the invention. A pharmaceutical composition of the invention comprises: an effective amount of a pharmaceutical agent selected from compounds of Formula (I) or Formula (II) and pharmaceutically acceptable salts, esters, amides, prodrugs, and active metabolites thereof; and a pharmaceutically acceptable excipient.

A “pharmaceutically acceptable excipient” refers to a substance that is not toxic, biologically intolerable, or otherwise biologically unsuitable for administration to a subject, such as an inert substance, added to a pharmacological composition or otherwise used as a vehicle, carrier, or diluent to facilitate administration of a pharmaceutical agent and that is compatible therewith. Examples of excipients include calcium carbonate, calcium phosphate, various sugars and types of starch, cellulose derivatives, gelatin, vegetable oils and polyethylene glycols.

Delivery forms of the pharmaceutical compositions containing one or more dosage units of the pharmaceutical agents may be prepared using suitable pharmaceutical excipients and compounding techniques known to those skilled in the art. The compositions may be administered in the inventive methods by oral, parenteral, rectal, topical, or ocular routes or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient mixed with pharmaceutically acceptable excipients such as inert diluents, disintegrating agents, binding agents, lubricating agents, sweetening agents, flavoring agents, coloring agents and preservatives agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol or water; preservatives (for example, methyl or propyl p-hydroxybenzoate or sorbic acid); wetting agents such as lecithin; and, if desired, flavoring or coloring agents.

The agents of this invention may also be administered by non-oral routes. For example, the compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of agent, admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Agents may alternatively be administered in methods of this invention by inhalation, via the nasal or oral routes, e.g., in a spray formulation also containing a suitable carrier.

Exemplary agents useful in methods of the invention will now be described by reference to the illustrative synthetic schemes for their general preparation below and the specific examples that follow. For the sake of brevity, the disclosures of the references cited below are herein incorporated by reference.

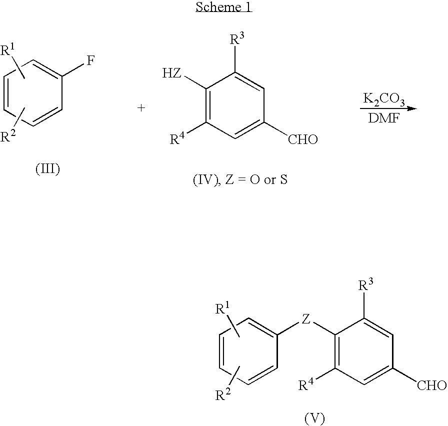

Aldehydes (V) where Z is O or S may be prepared by treatment of an aryl fluoride (III) with a phenol or thiol (IV), in the presence of a suitable base such as potassium carbonate or potassium tert-butoxide, in a polar, aprotic solvent such as DMF. The reaction may require heating to a temperature between about 50° C. and about 100° C. Aldehydes of formula (V) may then be transformed into compounds of Formula (I) as described in Schemes A and B below.

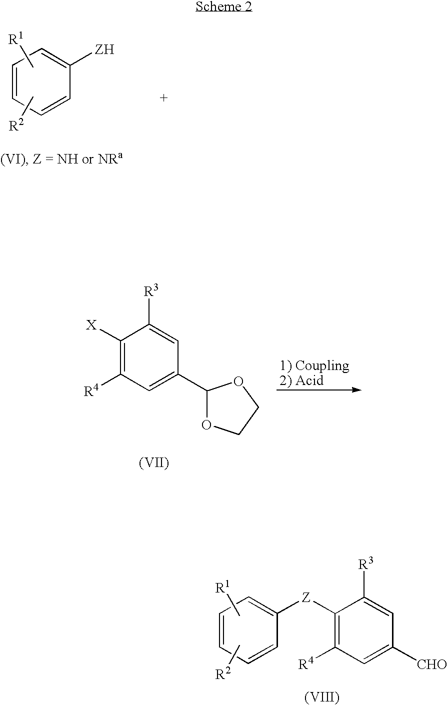

Amines of formula (VI) can be coupled with suitably protected benzene derivatives (VII), where X is bromide, chloride, iodide, triflate, or the like. Palladium catalysts such as Pd(OAc)₂ or Pd₂(dba)₃, or copper catalysts such as Cu(I)I or CuOAc, may be used. Optional additives include Cs₂CO₃, NaOtBu, K₃PO₄, dppf, and BINAP or other chelating phosphines. An exemplary solvent is toluene. The aldehyde protecting group, here shown as an acetal, may be removed under mild acidic conditions, such as p-toluenesulfonic acid, HCl, or camphorsulfonic acid. The resulting benzaldehydes of formula (VIII) may be transformed into compounds of formula (I) according to Schemes A and B.

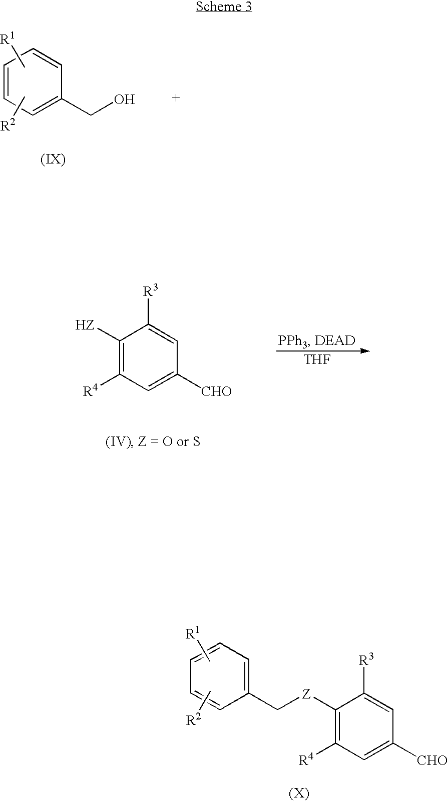

Intermediate aldehydes of formula (X) may be prepared by Mitsunobu reaction between a suitably substituted benzyl alcohol (IX) and the aldehyde (IV). Exemplary Mitsunobu conditions include triphenylphosphine and a dialkyl azodicarboxylate derivative such as diethyl or diisopropyl azodicarboxylate. A suitable solvent is THF. Aldehydes (X) may then be transformed into compounds of formula (I) as described in Schemes A and B.

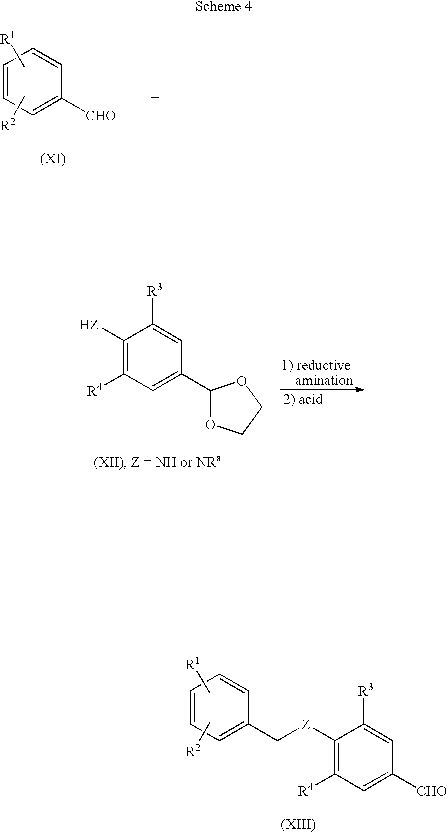

Aldehydes of formula (XI) may be converted into aldehydes of formula (XIII) by reaction with a suitably protected aniline (XII). Effective reducing agents include NaCNBH₃, NaBH₄, or Na(OAc)₃BH. Additives included mineral or Lewis acids such as acetic acid, HCl, or ZnCl₂. Suitable solvents include THF and toluene. The aldehyde protecting group may then be removed as described in Scheme 2. Aldehydes of formula (XIII) may be converted into compounds of formula (I) according to Scheme A or B.

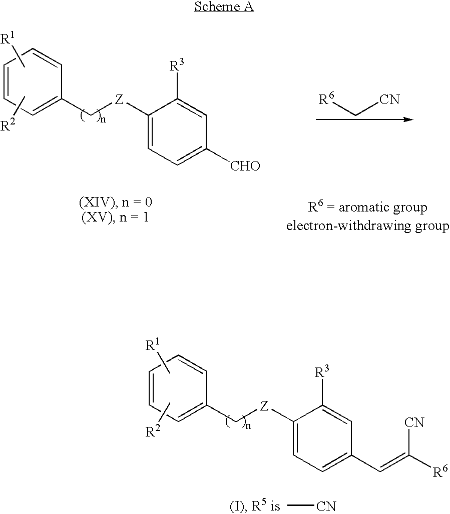

Referring to Scheme A, aldehydes of formula (XIV) or (XV) may be transformed into compounds of formula (I) wherein R⁵ is —CN as shown. Aldehydes (XIV) or (XV) are treated with an acetonitrile derivative, suitably substituted with R⁶ wherein R is an activating group such as an aromatic or electron-withdrawing group. The reaction is performed with the addition of a mild amine base, such as ammonium acetate or triethylamine, or with a hydroxide base, such as potassium hydroxide, in a solvent such as toluene or ethanol. The reaction may be performed at temperatures between about 0° C. and about 100° C.

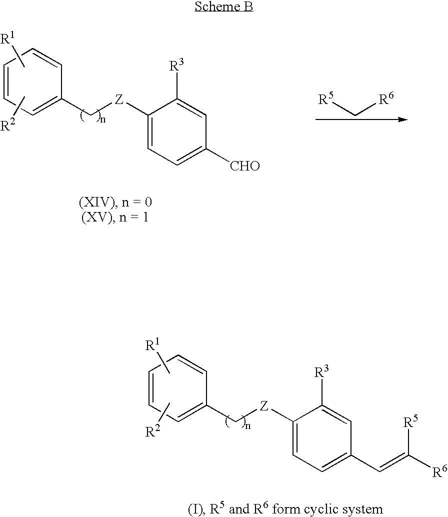

Referring to Scheme B, aldehydes of formula (XIV) or (XV) may be reacted with compounds in which R⁵ and R⁶ are taken together with the methylene group shown to form a 5-9 membered heterocycloalkyl or cycloalkyl moiety. The methylene group becomes the carbon of attachment for the ring system in the products of Formula (I). The reaction is performed in the presence of a suitable base such as ammonium acetate or sodium acetate, in a solvent such as acetonitrile or water, and at temperatures from about room temperature to about 100° C.

By following the above schemes, with routine adaptations, the following compounds may be prepared: 5-[2-Methoxy-3-(3-trifluoromethyl-phenoxy)-benzylidene]-thiazolidine-2,4-dione; 4-[4-(1,3-Dioxo-indan-2-ylidenemethyl)-2-methoxy-phenoxy]-2-trifluoromethyl-benzonitrile; 5-[4-(4-Nitro-3-trifluoromethyl-phenoxy)-benzylidene]-2-thioxo-imidazolidin-4-one; 4-[2-Methoxy-4-(3-methyl-5-oxo-1-phenyl-1,5-dihydro-pyrazol-4-ylidenemethyl)-phenoxy]-2-trifluoromethyl-benzonitrile; 4-[4-(1,3-Dioxo-indan-2-ylidenemethyl)-2-methoxy-phenoxy]-2-trifluoromethyl-benzoic acid methyl ester; 4-{4-[2-(5-Amino-4-cyano-1-phenyl-1H-pyrazol-3-yl)-2-cyano-vinyl]-2-methoxy-phenoxy}-2-trifluoromethyl-benzoic acid methyl ester; 4-[4-(2-Amino-4-oxo-4H-thiazol-5-ylidenemethyl)-2-methoxy-phenoxy]-2-trifluoromethyl-benzoic acid methyl ester; 4-[4-(1,3-Dioxo-indan-2-ylidenemethyl)-2-methoxy-phenoxy]-3-nitro-benzoic acid methyl ester; 4-[2-Methoxy-4-(5-oxo-1-phenyl-2-thioxo-imidazolidin-4-ylidenemethyl)-phenoxy]-2-trifluoromethyl-benzonitrile; 2-[4-(2-Chloro-4-fluoro-benzyloxy)-benzylidene]-indan-1,3-dione; 3-Cyclohexyl-5-[3-methoxy-4-(2-nitro-4-trifluoromethyl-phenoxy)-benzylidene]-thiazolidine-2,4-dione; 2-[3-Methoxy-4-(2-nitro-4-trifluoromethyl-phenoxy)-benzylidene]-malonic acid diethyl ester; and 4-[4-(2-Cyano-3-oxo-3-phenyl-propenyl)-2-methoxy-phenoxy]-3-nitro-benzoic acid methyl ester.

The following specific examples are provided to further illustrate the invention.

### EXAMPLES

Chemistry

In obtaining the characterization data described in the examples below, the following analytical protocols were followed as indicated.

### Protocol for Preparative Reversed-Phase HPLC

Waters® instrument
Column: Waters Xterra C-18, 5 μm, 19×50 mm
Flow rate: 30 mL/min
Detection: λ=254 nm

Gradient (acetonitrile/water, 0.1% formic acid)

| | | |
|---|---|---|
| 1) | 0.0 mm | 5% acetonitrile/95% water |
| 2) | 8.0 mm | 100% acetonitrile |

### Protocol for HPLC (Reversed-Phase)

Shimadzu instrument
Column: Princeton SPHER HTS, 5 μm, 3×50 mm
Flow rate: 2.2 mL/min

Detection: Sedex 75 ELS coupled to Finnigan AQA electrospray mass spectrometer Gradient (acetonitrile/water, 0.1% trifluoroacetic acid)

| | | |
|---|---|---|
| 1) | 0.0 mm | 0.1% acetonitrile/99.9% water |
| 2) | 8.0 mm | 100% acetonitrile |

Mass spectra were obtained on a Finnigan AQA using electrospray ionization (ESI) in either positive or negative modes as indicated.

NMR spectra were obtained on a Varian model VXR-300S (300 MHz) spectrometer. The format of the ¹H NMR data below is: chemical shift in ppm down field of the tetramethylsilane reference (multiplicity, coupling constant J in Hz, integration).

Where solutions are “concentrated” they are generally concentrated under reduced pressure using a rotary evaporator.
Preparation (a).

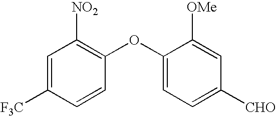

### 3-Methoxy-4-(2-nitro-4-trifluoromethyl-phenoxy)benzaldehyde

To a solution of vanillin (1.1 g, 7.7 mmol) and 4-fluoro-3-nitro-benzotrifluoride (0.85 mL, 6.0 mmol) in DMF (10 mL) was added K₂CO₃ (1.70 g, 12.46 mmol) at ambient temperature (rt). The mixture was heated to 80° C. and stirred for 12 h. The mixture was concentrated and the residue was diluted with ethyl acetate (EtOAc). The suspension was washed sequentially with water, brine, dried with Na₂SO₄, and concentrated to yield a pale yellow liquid, which solidified after standing at rt. After triturating with Et₂O-hexanes (1:3) the solid product was filtered and dried under reduced pressure (1.7 g, 83%).

### Example 1

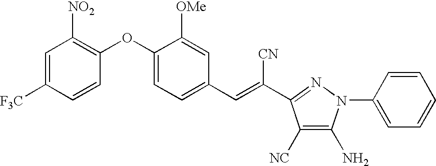

### 5-Amino-3-{1-cyano-2-[3-methoxy-4-(2-nitro-4-trifluoromethyl-phenoxy)-phenyl]vinyl}-1-phenyl-1H-pyrazole-4-carbonitrile

Anhydrous ammonium acetate (0.16 g, 2.0 mmol) was added to a solution of 3-methoxy-4-(2-nitro-4-trifluoromethyl-phenoxy)benzaldehyde (0.51 g, 1.5 mmol) (Preparation (a)), and 5-amino-4-cyano-1-phenyl-3-pyrazoleacetonitrile (0.40 g, 1.8 mmol) in 10 mL anhydrous toluene at rt and the mixture was slowly heated to 90° C. After stirring overnight at 90° C., the mixture was cooled to rt, filtered through diatomaceous earth and washed with EtOAc (2×15 mL). The combined filtrate was washed sequentially with water, dried with Na₂SO₄ and concentrated to yield a semi-solid product, which solidified on standing at rt. Recrystallization from methanol (MeOH)—CHCl₃ yielded the desired product as a colorless solid (0.52 g, 68%). ¹H NMR (CDCl₃): 3.82 (s, 3H), 3.96 (br s, 2H), 6.96 (d, 1H), 7.24 (d, 1H), 7.52 (m, 5H), 7.70 (m, 3H), 8.28 (d, 1H), 9.10 (s, 1H). LCMS (ESI): RT 2.10 min, purity 98%, [M+1] 547.

The compounds of Examples 2-6 were prepared in a manner similar to that described in Example 1.

### Example 2

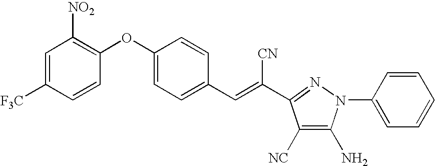

### 5-Amino-3-{1-cyano-2-[4-(2-nitro-4-trifluoromethyl-phenoxy)-phenyl]-vinyl}-1-phenyl-1H-pyrazole-4-carbonitrile

¹H NMR (CDCl₃): 3.94 (br s, 2H), 7.18 (d, 2H), 7.24 (d, 1H), 7.48 (m, 3H), 7.67 (m, 2H), 7.85 (m, 1H), 7.98 (m, 2H), 8.31 (d, 1H), 9.10 (s, 1H). LCMS (ESI): RT 1.99 min, purity 100%, [M+1] 517.

### Example 3

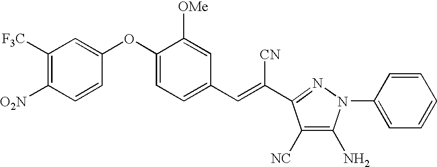

### 5-Amino-3-{1-cyano-2-[3-methoxy-4-(4-nitro-3-trifluoromethyl-phenoxy)-phenyl]-vinyl}-1-phenyl-1H-pyrazole-4-carbonitrile

¹H NMR (CDCl₃): 3.86 (s, 3H), 4.72 (br s, 2H), 7.06 (m, 1H), 7.22 (d, 1H), 7.38 (d, 1H), 7.55 (m, 6H), 7.83 (d, 1H), 7.97 (m, 2H). LCMS (ESI): RT 2.06 min, purity 100%, [M+1] 547.

### Example 4

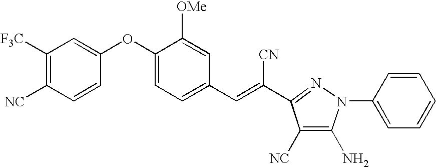

### 5-Amino-3-{1-cyano-2-[4-(4-cyano-3-trifluoromethyl-phenoxy)-3-methoxy-phenyl]-vinyl}-1-phenyl-1H-pyrazole-4-carbonitrile

¹H NMR (CDCl₃): 3.88 (s, 3H), 4.72 (br s, 2H), 7.07 (m, 1H), 7.19 (d, 1H), 7.34 (d, 1H), 7.52 (m, 2H), 7.58 (m 4H), 7.76 (d, 1H), 7.82 (d, 1H), 7.99 (s, 1H). LCMS (ESI): RT 2.02 min, purity 100%, [M+1] 527.

### Example 5

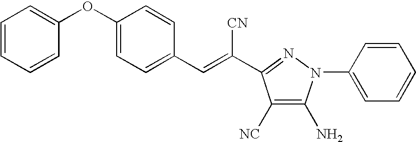

### 5-Amino-3-[1-cyano-2-(4-phenoxy-phenyl)-vinyl]-1-phenyl-1H-pyrazole-4-carbonitrile

¹H NMR (CDCl₃): 4.70 (br s, 2H), 7.06 (m, 4H), 7.21 (m, 1H), 7.41 (m, 2H), 7.49 (m, 1H), 7.56 (m, 4H), 7.95 (m, 3H). LCMS (ESI): RT 1.99 min, purity 100%, [M+1] 404.

### Example 6

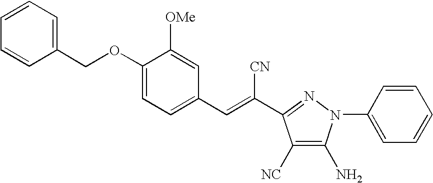

### 5-Amino-3-[2-(4-benzyloxy-3-methoxy-phenyl)-1-cyano-vinyl]-1-phenyl-1H-pyrazole-4-carbonitrile

¹H NMR (CDCl₃): 3.98 (s, 3H), 4.66 (br s, 2H), 5.24 (s, 2H), 6.94 (d, 1H), 7.42 (m, 7H), 7.56 (m, 4H), 7.75 (d, 1H), 7.89 (s, 1H). LCMS (ESI): RT 1.98 min, purity 99%, [M+1] 448.

### Example 7

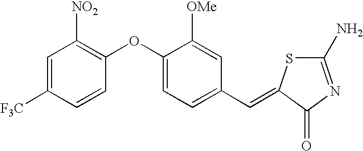

### 2-Amino-5-[3-methoxy-4-(2-nitro-4-trifluoromethyl-phenoxy)-benzylidene]-thiazol-4-one

Anhydrous ammonium acetate (0.30 g, 3.9 mmol) was added to a solution of 3-methoxy-4-(2-nitro-4-trifluoromethyl-phenoxy)benzaldehyde (0.34 g, 1.0 mmol) and 2-aminothiazol-4-one (0.14 g, 1.2 mmol) in 10 mL ethanol at rt. The reaction mixture was stirred overnight at reflux and then cooled to rt. The reaction mixture was concentrated and a few mL of water was added with trituration. The precipitate was collected by filtration, washed with cold water and then resuspended in Et₂O with trituration. The pale yellow product was collected by filtration, washed with cold Et₂O and dried in vacuo (0.43 g, 98%). ¹H NMR (DMSO-d₆): 3.81 (s, 3H), 7.08 (d, 1H), 7.28 (m, 1H), 7.46 (m, 2H), 7.66 (s, 1H), 7.95 (m, 1H), 8.46 (d, 1H). LCMS (ESI): RT 1.43 min, purity 99%, [M+1] 440.

The compounds of Examples 8-9 were prepared a manner similar to that described in Example 7.

### Example 8

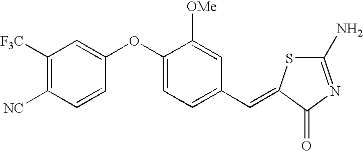

### 4-[4-(2-Amino-4-oxo-4H-thiazol-5-ylidenemethyl)-2-methoxy-phenoxy]-2-trifluoromethyl-benzonitrile

¹H NMR (CDCl₃/DMSO-d₆): 3.6 (s, 3H), 6.84 (m, 1H), 6.97 (m, 3H), 7.08 (d, 1H), 7.47 (s, 1H), 7.56 (d, 1H). LCMS (ESI): RT 1.37 min, purity 100%, [M+CH₃CN+1] 461.

### Example 9

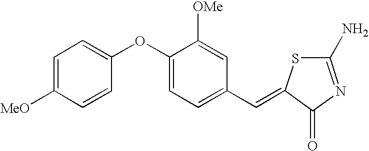

### 2-Amino-5-[4-(4-methoxy-phenoxy)-benzylidene]-thiazol-4-one

¹H NMR (DMSO-d₆): 3.77 (s, 3H), 7.05 (m, 6H), 7.56 (m, 3H). LCMS (ESI): RT 1.30 min, purity 93%, [M+1] 327.

### Example 10

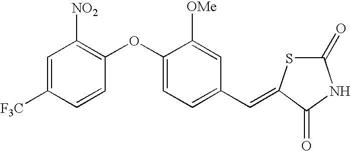

### 5-[3-Methoxy-4-(2-nitro-4-trifluoromethyl-phenoxy)-benzylidene]-thiazolidine-2,4-dione

A mixture of 3-methoxy-4-(2-nitro-4-trifluoromethyl-phenoxy)benzaldehyde (0.34 g, 1.0 mmol), 2,4-thiazolidinedione (0.14 g, 1.2 mmol) and sodium acetate (0.20 g, 2.5 mmol) in 10 mL acetonitrile was heated to 100° C. After stirring for 45 minutes the mixture was cooled to rt. Water (10 mL) was added and the mixture was heated to 75° C. After stirring for 10 min the mixture was cooled to rt and the solid product was filtered and washed with water. The crude product was dissolved in acetone, filtered and concentrated. Trituration with Et₂O yielded the desired product as colorless powder (yield 0.32 g, 73%). ¹H NMR (DMSO-d₆): 3.80 (s, 3H), 7.08 (d, 1H), 7.28 (m, 1H), 7.43 (d, 1H), 7.52 (d, 1H), 7.83 (s, 1H), 7.96 (m, 1H), 8.46 (d, 1H). LCMS (ESI): RT 1.72 min, purity 100%, [2M+H₂O] 898.

The products of Examples 11-20 were prepared in a manner similar to that described in Example 10.

### Example 11

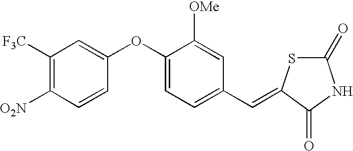

### 5-[3-Methoxy-4-(4-nitro-3-trifluoromethyl-phenoxy)-benzylidene]-thiazolidine-2,4-dione

¹H NMR (DMSO-d₆): 3.80 (s, 3H), 7.26 (m, 2H), 7.43 (d, 1H), 7.55 (m, 2H), 7.84 (s, 1H), 8.16 (d, 1H). LCMS (ESI): RT 1.75 min, purity 94%, did not ionize.

### Example 12

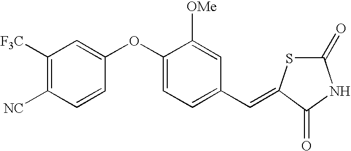

### 4-[4-(2,4-Dioxo-thiazolidin-5-ylidenemethyl)-2-methoxy-phenoxy]-2-trifluoromethyl-benzonitrile

¹H NMR (DMSO-d₆): 3.80 (s, 3H), 7.25 (m, 2H), 7.38 (d, 1H), 7.52 (m, 2H), 7.72 (s, 1H), 8.09 (d, 1H). LCMS (ESI): RT 1.66 min, purity 99%, [2M+H₂O] 858.

### Example 13

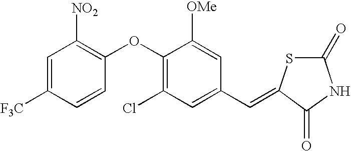

### 5-[3-Chloro-5-methoxy-4-(2-nitro-4-trifluoromethyl-phenoxy)-benzylidene]-thiazolidine-2,4-dione

¹H NMR (DMSO-d₆): 3.84 (s, 3H), 7.06 (d, 1H), 7.46 (s, 2H), 7.78 (s, 1H), 7.95 (m, 1H), 8.50 (d, 1H). LCMS (ESI): RT 1.86 min, purity 95%, did not ionize.

### Example 14

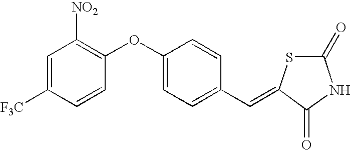

### 5-[4-(2-Nitro-4-trifluoromethyl-phenoxy)-benzylidene]-thiazolidine-2,4-dione

¹H NMR (CDCl₃): 7.12 (m, 3H), 7.48 (m, 2H), 7.70 (s, 1H), 7.74 (m, 1H), 8.2 (d, 1H). LCMS (ESI): RT 1.67 min, purity 100%, [M+CH₃CN+1] 452.

### Example 15

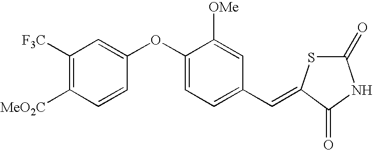

### 4-[4-(2,4-Dioxo-thiazolidin-5-ylidenemethyl)-2-methoxy-phenoxy]-2-trifluoromethyl-benzoic acid methyl ester

¹H NMR (CDCl₃): 3.86 (s, 3H), 3.93 (s, 3H), 7.05 (m, 1H), 7.15 (m, 3H), 7.32 (d, 1H), 7.83 (m, 2H). LCMS (ESI): RT 1.65 min, purity 95%, [2M+1] 907.

### Example 16

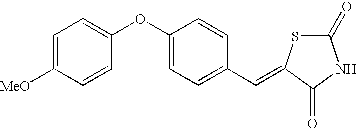

### 5-[4-(4-Methoxy-phenoxy)-benzylidene]-thiazolidine-2,4-dione

¹H NMR (CDCl₃): 3.76 (s, 3H), 6.92 (m, 6H), 7.37 (m, 2H), 7.68 (s, 1H). LCMS (ESI): RT 1.59 min, purity 97%, [M+CH₃CN+1] 369.

### Example 17

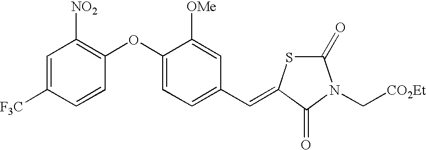

### {5-[3-Methoxy-4-(2-nitro-4-trifluoromethyl-phenoxy)-benzylidene]-2,4-dioxo-thiazolidin-3-yl}-acetic acid ethyl ester

¹H NMR (CDCl₃): 1.32 (t, 3H), 3.86 (s, 3H), 4.25 (q, 2H), 4.50 (s, 2H), 6.94 (d, 1H), 7.22 (m, 3H), 7.70 (m, 1H), 7.92 (s, 1H), 8.27 (d, 1H). LCMS (ESI): RT 1.94 min, purity 100%, [M+1] 527.

### Example 18

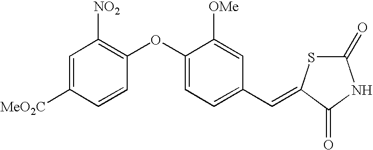

### 4-[4-(2,4-Dioxo-thiazolidin-5-ylidenemethyl)-2-methoxy-phenoxy]-3-nitro-benzoic acid methyl ester

¹H NMR (DMSO-d₆): 3.78 (s, 3H), 3.88 (s, 3H), 7.0 (d, 1H), 7.28 (m, 1H), 7.43 (d, 1H), 7.50 (d, 1H), 7.85 (s, 1H), 8.32 (m, 1H), 8.52 (d, 1H). LCMS (ESI): RT 1.61 min, purity 99%, did not ionize.

### Example 19

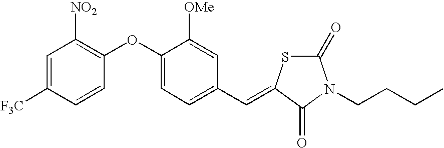

### 3-Butyl-5-[3-methoxy-4-(2-nitro-4-trifluoromethyl-phenoxy)-benzylidene]-thiazolidine-2,4-dione

¹H NMR (DMSO-d₆): 0.98 (t, 3H), 1.38 (m, 2H), 1.68 (m, 2H), 3.80 (m, 5H), 6.93 (d, 1H), 7.21 (m, 3H), 7.70 (m, 1H), 7.87 (s, 1H), 8.27 (d, 1H). LCMS (ESI): RT 2.06 min, purity 100%, did not ionize.

### Example 20

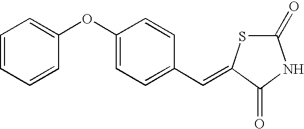

### 5-(4-Phenoxy-benzylidene)-thiazolidine-2,4-dione

¹H NMR (DMSO-d₆): 7.1 (m, 4H), 7.23 (m, 1H), 7.45 (m, 2H), 7.62 (m, 2H), 7.72 (s, 1H). LCMS (ESI): RT 1.65 min, purity 100%, [2M+1] 595.

### Example 21

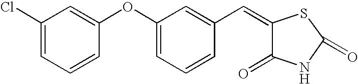

### 5-[3-(3-Chloro-phenoxy)-benzylidene]-thiazolidine-2,4-dione

A mixture of 3-(3-chloro-phenoxy)benzaldehyde (0.19 mL, 0.9 mmol), 2,4-thiazolidinedione (0.12 g, 1.0 mmol) and sodium acetate (0.16 g, 2.0 mmol) was heated to 100° C. under stirring. After 45 min, the mixture was cooled to 50° C. and carefully diluted with water (10 mL). After stirring for 10 min, the mixture was cooled to rt and the solid product was filtered and washed with water. The crude product was recrystallized from 1:1 Et₂O-hexanes (0.27 g, 81%). ¹H NMR (DMSO-d₆): 7.21 (m, 3H), 7.23 (m, 1H), 7.37 (m, 1H), 7.52 (m, 3H), 7.75 (s, 1H). LCMS (ESI): RT 1.78 min, purity 100%, [2M+1] 663.

### Example 22

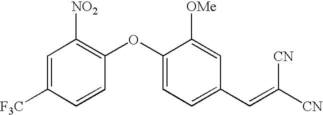

### 2-[3-Methoxy-4-(2-nitro-4-trifluoromethyl-phenoxy)-benzylidene]-malononitrile

Ammonium acetate (0.082 g, 1.0 mmol) was added to a solution of 3-methoxy-4-(2-nitro-4-trifluoromethyl-phenoxy)benzaldehyde (0.34 g, 1.0 mmol) and malononitrile (0.080 g, 1.2 mmol) in dry toluene (10 mL) at rt. After stirring for 16 h, the mixture was filtered through diatomaceous earth, washing with toluene (2×10 mL). The combined filtrate was washed with brine, dried and concentrated. Trituration with hexanes resulted a pale yellow solid, which was filtered and dried under reduced pressure (0.29 g, 75%). ¹H NMR (CDCl₃): 3.87 (s, 3H), 7.0 (d, 1H), 7.20 (d, 1H), 7.44 (m, 1H), 7.76 (m, 3H), 8.30 (d, 1H). LCMS (ESI): RT 1.73 min, purity 100%, did not ionize.

The compounds of Examples 23 and 24 were prepared in a manner similar to that described in Example 22.

### Example 23

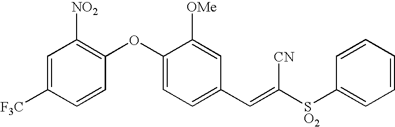

### 2-Benzenesulfonyl-3-[3-methoxy-4-(2-nitro-4-trifluoromethyl-phenoxy)-phenyl]-acrylonitrile

¹H NMR (CDCl₃): 3.84 (s, 3H), 6.96 (d, 1H), 7.20 (d, 1H), 7.50 (m, 1H), 7.65 (m, 2H), 7.73 (m, 3H), 8.04 (m, 2H), 8.21 (s, 1H), 8.28 (d, 1H). LCMS (ESI): RT 1.98 min, purity 100%, [M+H₂O] 522.

### Example 24

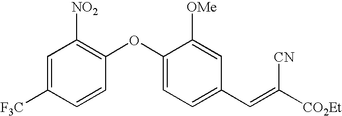

### 2-Cyano-3-[3-methoxy-4-(2-nitro-4-trifluoromethyl-phenoxy)-phenyl]-acrylic acid ethyl ester

¹H NMR (CDCl₃): 1.44 (t, J=7.2 Hz, 3H), 3.86 (s, 3H), 4.42 (q, 2H), 6.97 (d, 1H), 7.22 (d, 1H), 7.52 (m, 1H), 7.72 (m, 1H), 7.88 (d, 1H), 8.23 (s, 1H), 8.29 (d, 1H). LCMS (ESI): RT 1.85 min, purity 100%, [M+H₂O] 454.

### Example 25

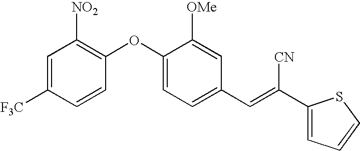

### 3-[3-Methoxy4-(2-nitro4-trifluoromethyl-phenoxy)-phenyl]-2-thiophen-2-yl-acrylonitrile

To a solution of 3-methoxy-4-(2-nitro-4-trifluoromethyl-phenoxy)benzaldehyde (0.15 g, 0.44 mmol) and thiophen-2-yl-acetonitrile (0.047 mL, 0.44 mmol) in absolute ethanol (1.5 mL) at 0° C. was added 50% KOH (0.2 mL). The mixture was slowly warmed to rt and stirred for 30 min. After addition of water the solid product was filtered. Recrystallization from Et₂O yielded a colorless crystalline product (0.098 g, 55%). ¹H NMR (CDCl₃): 3.80 (s, 3H), 6.95 (d, 1H), 7.11 (m, 1H), 7.22 (d, 1H), 7.38 (m, 4H), 7.71 (m, 2H), 8.27 (d, 1H). LCMS (ESI): RT 2.06 min, purity 100%, [M+H₂O] 464.

The compounds of Examples 26 and 27 were prepared in a manner similar to that described in Example 25.

### Example 26

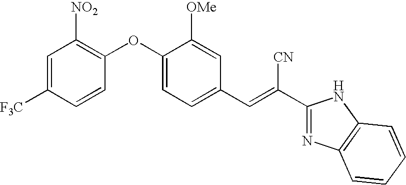

### 2-(1H-Benzoimidazol-2-yl)-3-[3-methoxy-4-(2-nitro-4-trifluoromethyl-phenoxy)-phenyl]-acrylonitrile

¹H NMR (CDCl₃): 3.62 (s, 3H), 6.75 (d, 1H), 7.0 (m, 3H), 7.29 (m, 1H), 7.38 (m, 2H), 7.5 (m, 1H), 7.64 (d, 1H), 8.0 (d, 1H), 8.11 (s, 1H). LCMS (ESI): RT 1.63 min, purity 100%, [M+CH₃CN+1] 522.

### Example 27

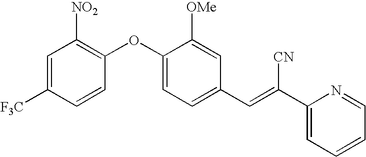

### 3-[3-Methoxy-4-(2-nitro-4-trifluoromethyl-phenoxy)-phenyl]-2-pyridin-2-yl-acrylonitrile

¹H NMR (CDCl₃): 3.84 (s, 3H), 6.96 (d, 1H), 7.24 (d, 1H), 7.43 (m, 2H), 7.58 (s, 1H), 7.70 (m, 1H), 7.80 (d, 1H), 8.0 (m, 1H), 8.28 (d, 1H), 8.68 (dd, 1H), 8.96 (m, 1H). LCMS (ESI): RT 1.61 min, purity 98%, [M+CH₃CN+1] 483.

### Example 28

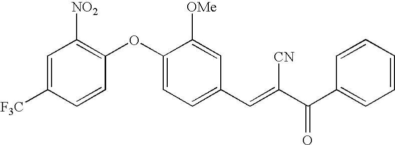

### 2-Benzoyl-3-[3-methoxy-4-(2-nitro-4-trifluoromethyl-phenoxy)-phenyl]-acrylonitrile

Triethylamine (0.1 mL) was added to a solution of 3-methoxy-4-(2-nitro-4-trifluoromethyl-phenoxy)benzaldehyde (0.15 g, 0.44 mmol) and 3-oxo-3-phenyl-propionitrile (0.064 g, 0.44 mmol) in absolute ethanol (1.5 mL) at rt. The mixture was stirred at 80° C. for 30 min and then was cooled to rt. The solid product was isolated after addition of cold 5% aq. HCI. The product was isolated by flash chromatography (eluting with 25% EtOAc/hexanes) as a colorless solid (0.14 g, 70%). ¹H NMR (CDCl₃): 3.88 (s, 3H), 6.9 (d, 1H), 7.24 (d, 1H), 7.55 (m, 3H) 7.70 (m, 2H), 7.74 (m, 3H), 8.05 (s, 1H), 8.28 (d, 1H). LCMS (ESI): RT 1.98 min, purity 92%, [M+1] 469.

### Example 29

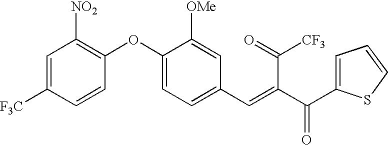

### 4,4,4-Trifluoro-2-[3-methoxy-4-(2-nitro-4-trifluoromethyl-phenoxy)-benzylidene]-1-thiophen-2-yl-butane-1,3-dione

The title compound was prepared in a manner similar to that described in Example 30, using piperidine as base. ¹H NMR (CDCl₃): 3.88 (s, 3H), 6.92 (d, 1H), 7.22 (m, 2H), 7.36 (m, 2H), 7.70 (m, 2H), 7.84 (d, 1H), 7.90 (d, 1H), 8.26 (d, 1H). LCMS (ESI): RT 1.93 min, purity 99%, did not ionize.

### Example 30

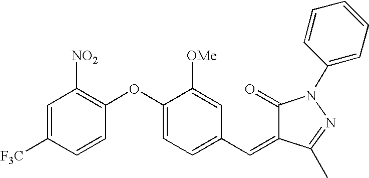

### 4-[3-Methoxy-4-(2-nitro-4-trifluoromethyl-phenoxy)-benzylidene]-5-methyl-2-phenyl-2,4-dihydro-pyrazol-3-one

Ammonium acetate (0.082 g, 1.0 mmol) was added to a solution of 3-methoxy-4-(2-nitro-4-trifluoromethyl-phenoxy)benzaldehyde (0.15 g, 0.44 mmol) and 5-methyl-2-phenyl-2,4-dihydro-pyrazol-3-one (0.077 g, 0.44 mmol) in dry acetonitrile (2 mL) at rt. After stirring for 6 h, the mixture was filtered through diatomaceous earth, washing with EtOAc (2×10 mL). The combined filtrate was concentrated. The residue was diluted with EtOAc, washed with brine, dried and concentrated. Trituration with Et₂O-hexanes (1:1) gave a pale yellow solid, which was filtered and dried under reduced pressure (0.16 g, 75%). ¹H NMR (CDCl₃): 2.39 (s, 3H), 3.94 (s, 3H), 6.98 (d, 1H), 7.21 (m, 2H), 7.36 (s, 1H), 7.44 (m, 2H), 7.68 (m, 2H), 7.94 (m, 2H), 8.28 (d, 1H), 9.11 (d, 1H). LCMS (ESI): RT 2.14 min, purity 98%, [M+1] 498.

Compounds 31-36 were prepared in a manner similar to that described in Example 30.

### Example 31

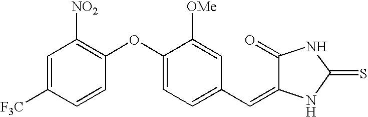

### 5-[3-4-(2-nitro-4-trifluoromethyl-phenoxy)-benzylidene]-2-thioxo-imidazolidin-4 4-one

¹NMR (DMSO-d₆): 3.82 (s, 3H), 6.55 (s, 1H), 7.02 (d, 1H), 7.33 (d, 1H), 7.48 (m, 2H), 7.94 (m, 1H), 8.45 (d, 1H). LCMS (ESI): RT 1.79 min, purity 97%, [M+1] 440.

### Example 32

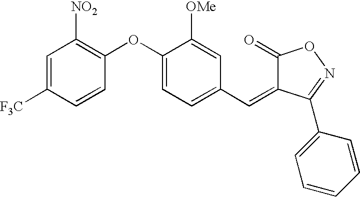

### 4-[3-4-(2-nitro-4-trifluoromethyl-phenoxy)-benzylidene]-3-phenyl-4H-isoxazol-5-one

¹H NMR (CDCl₃): 3.92 (s, 3H), 7.0 (d, 1H), 7.17 (d, 1H), 7.58 (m, 7H), 7.74 (m, 1H), 8.29 (d, 1H), 8.80 (d, 1H). LCMS (ESI): RT 1.98 min, purity 93%, [M+1] 485.

### Example 33

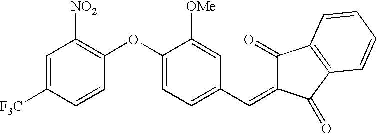

### 2-[3-4-(2-nitro-4-trifluoromethyl-phenoxy)-benzylidene]-indan-1,3-dione

¹H NMR (CDCl₃): 3.98 (s, 3H), 7.0 (d, 1H), 7.23 (d, 1H), 7.74 (m, 2H), 7.87 (m, 3H), 8.05 (m, 2H), 8.29 (d, 1H), 8.90 (d, 1H). LCMS (ESI): RT 1.99 min, purity 97%, [M+1] 470.

### Example 34

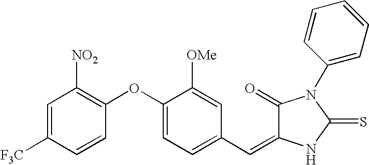

### 5-[3-Methoxy-4-(2-nitro-4-trifluoromethyl-phenoxy)-benzylidene]-3-phenyl-2-thioxo-imidazolidin-4-one

¹H NMR (DMSO-d₆): 3.85 (s, 3H), 6.74 (s, 1H), 7.04 (d, 1H), 7.39 (m, 3H), 7.54 (m, 5H), 7.98 (m, 1H), 8.47 (d, 1H). LCMS (ESI): RT 1.90 min, purity 99%, [M+1] 516.

### Example 35

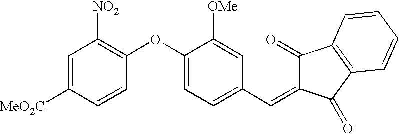

### 4-[4-(1,3-Dioxo-indan-2-ylidenemethyl)-2-methoxy-phenoxy]-3-nitro-benzoic acid methyl ester

¹H NMR (DMSO-d₆): 3.88 (s, 6H), 7.10 (d, 1H), 7.44 (d, 1H), 7.91 (s, 1H), 8.02 (m, 4H), 8.16 (m, 2H), 8.55 (d, 1H), 8.75 (d, 1H). LCMS (ESI): RT 1.91 min, purity 99%, [M+CH₃CN+1] 501.

### Example 36

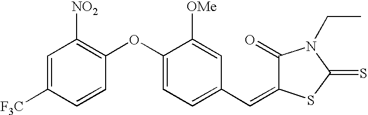

### 3-Ethyl-5-[3-methoxy-4-(2-nitro-4-trifluoromethyl-phenoxy)-benzylidene]-2-thioxo-thiazolidin-4-one

¹H NMR (CDCl₃): 1.32 (t, 3H), 3.86 (s, 3H), 4.22 (q, 2H), 6.95 (d, 1H), 7.12 (d, 1H), 7.18 (m, 1H), 7.24 (d, 1H), 7.71 (m, 2H), 8.27 (d, 1H). LCMS (ESI): RT 2.25 min, purity 99%, [M+1] 485.

### Example 37

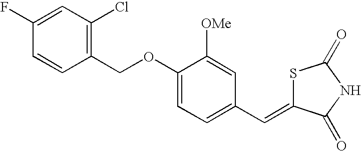

### 5-[4-(2—Chloro-4-fluoro-benzyloxy)-3-methoxy-benzylidene]-thiazolidine-2,4-dione

Step A. A solution of diethyl azodicarboxylate (0.75 mL, 4.4 mmol) in dry THF (10 mL) was added slowly to a solution of Ph₃P (1.15 g, 4.45 mmol), vanillin (0.61 g, 4.0 mmol) and 2-chloro-4-fluorobenzyl alcohol (0.64 g, 4.0 mmol) in THF (15 mL) at 0° C. under argon. After the addition was complete, the mixture was slowly warmed to rt and stirred for 16 h. The mixture was concentrated and the residue purified by flash chromatography. Elution with 10% acetone in CH₂Cl₂ yielded 4-(2-chloro-4-fluoro-benzyloxy)-3-methoxy-benzaldehyde as colorless solid.

Step B. A mixture of aldehyde from Step A (0.70 g, 2.3 mmol), 2,4-thiazolidinedione (0.27 g, 2.3 mmol) and sodium acetate (0.47 g, 5.0 mmol) was heated to 90° C. and stirred at that temperature for 45 min. The mixture was cooled to rt, water (10 mL) was added, and the mixture was heated to 80° C. and stirred for 10 min. After cooling to rt, the solids were collected by filtration and washed with water. The crude product was dissolved in acetone, filtered, and the filtrate was concentrated. Trituration with Et₂O produced the desired product as colorless solid (0.48 g, 35% for 2 steps). ¹H NMR (DMSO-d₆): 3.80 (s, 3H), 5.15 (s, 2H), 7.16 (m, 3H), 7.28 (m, 1H), 7.41 (s, 1H), 7.53 (dd, 1H), 7.65 (m, 1H). LCMS (ESI): RT 0.87 min, purity 100%, did not ionize.

Compounds 38, 39, and 40 were prepared in a manner similar to that described in Example 37.

### Example 38

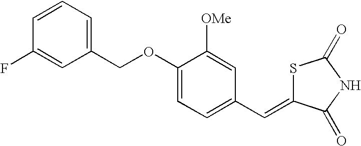

### 5-[4-(3-Fluoro-benzyloxy)-3-methoxy-benzylidene]-thiazolidine-2,4-dione

¹H NMR (DMSO-d₆): 3.83 (s, 3H), 5.20 (s, 2H), 7.24 (m, 6H), 7.45 (m, 1H), 7.74 (s, 1H). LCMS (ESI): RT 1.43 min, purity 98%, [2M+1] 719.

### Example 39

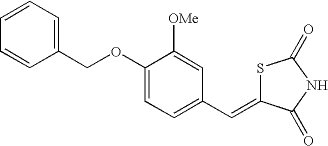

### 5-(4-Benzyloxy-3-methoxy-benzylidene)-thiazolidine-2,4-dione

¹H NMR (DMSO-d₆): 3.81 (s, 3H), 5.15 (s, 2H), 7.19 (m, 3H), 7.42 (m, 5H), 7.73 (s, 1H). LCMS (ESI): RT 1.58 min, purity 99%, [M+CH₃CN+1] 383.

### Example 40

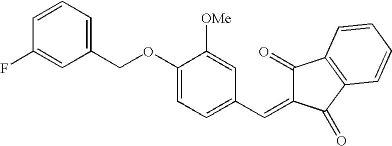

### 2-[4-(3-Fluoro-benzyloxy)-3-methoxy-benzylidene]-indan-1,3-dione

¹H NMR (DMSO-d₆): 3.95 (s, 3H), 5.30 (s, 2H), 7.28 (m, 4H), 7.48 (m, 1H), 7.82 (s, 1H), 7.98 (m, 5H), 8.72 (s, 1H). LCMS (ESI): RT 1.72 min, purity 100%, [M+1] 389.

Biological Data

The ligand binding domain of ERR-α (aa 183-424) was subcloned in frame with the N-terminal His tag and the cleavable thrombin site in pET28. The protein was expressed in BL21(DE3⁺) following induction with 100 μM IPTG at 16° C. Cells were harvested after 16 hours (h) of induction and lysed in 20 mM Tris pH 7.5, 500 mM NaCl, 5 mM imidazole, 5% glycerol, protease inhibitor cocktail (-EDTA) and 2 mM β-ME. Insoluble material was removed by centrifugation at 40,000×g for 1 hr. Clarified homogenate was applied on a Ni-NTA column and after applying an imidazole gradient the protein was eluted. The protein was further purified on size exclusion chromatography to an apparent homogeneity of 95% as judged by SDS-PAGE chromatography.

Binding affinities of compounds were determined by screening against the ligand binding domain of ERR-α using ThermoFluor® technology (U.S. Pat. No. 6,020,141 and U.S. Pat. No. 6,036,920, and Journal of Biomolecular Screening 6 (6), 2002, pgs 429-440). Assay plates were prepared by dispensing 2 μL of a protein-dye solution and 2 μL of the test compound in a 384-well plate. The conditions used in the screen were: 0.1 mg/mL ERR-α, 25 mM Na-phosphate buffer at pH 7.6, 200 mM NaCl, 10% glycerol, 16 μM ANS, 2% DMSO and the final concentration of test compound was 100 μM. Finally 1 μL of mineral oil was dispensed on top to prevent evaporation during the high throughput screen (HTS). ThermoFluor® is an HTS assay that measures protein unfolding based on fluorescence detection of the denatured form of the protein. The reporter for the protein unfolding event is the environmentally sensitive dye ANS that is incorporated in the screening buffer. During a typical experiment the 384-well plate is heated at a ramping rate of 1° C./min and the thermal unfolding of the protein is monitored at 1° C. intervals by measuring fluorescence changes detected through a CCD camera. Captured images are integrated and a melting curve is generated that relates fluorescence to fraction of unfolded protein as a function of temperature. For the ERR-α screen, data were collected from 30 to 80° C. at 1° C. intervals and the protein melted under the screening conditions with a characteristic melting temperature, Tₘ, of 52.1° C. Hits were identified from the screen by measuring an increase in the melting temperature of the protein.

In order to estimate binding affinities, it was taken into account that the oligomeric state of ERR-α, which is a dimer (N₂), and that of a single ligand (L_{f}) can interact per monomer subunit with equal affinity. The melting curve for such a system is described by the following three equilibria:

$$\begin{matrix}{N}_{2} {\to }^{{K}_{u}} 2 U & ( 1 ) \\ {N}_{2} L {\to }^{{K}_{d 1}} {N}_{2} + {L}_{f} & ( 2 ) \\ {N}_{2} {L}_{2} {\to }^{{K}_{d 1}} {N}_{2} L + {L}_{f} & ( 3 )\end{matrix}$$

The first equilibrium describes the denaturation of ERR-α dimers; the second equilibrium describes the dissociation of the first ligand from the single ligand occupied ERR-α dimers (N₂L); and the third equilibrium describes the dissociation of the second ligand from the fully occupied ERR-α dimers (N₂L₂).

Following the derivations of Brandts and Lin (Biochemistry, 29, 6967, 1990) the dissociation constants for the ligands (K_{d1}^{t}^{ₘ}) can be determined at T=Tₘ for any ligand concentration Lₜ by solving numerically the conservation of mass equations:

$$\begin{matrix}{P}_{t} = 2 \times {N}_{2} + 2 \times {N}_{2} L + 2 \times {N}_{2} {L}_{2} + U & ( 4 ) \\ {L}_{t} = {N}_{2} L + 2 \times {N}_{2} {L}_{2} + {L}_{f} \\ and & ( 5 ) \\ {L}_{f} = \frac{- b + \sqrt{{b}^{2} - 4 \times a \times c + 2 \times c \times {P}_{t}}}{2 \times c} \\ where & ( 6 ) \\ a = \frac{{P}_{t}^{2}}{2 \times {K}_{u}} & ( 7 ) \\ b = \frac{{P}_{t}^{2}}{2 \times {K}_{u} \times {K}_{d 1}^{{T}_{m}}} & ( 8 ) \\ c = \frac{{P}_{t}^{2}}{2 \times {K}_{u} \times {K}_{d 1}^{{T}_{m}} \times {K}_{d 1}^{{T}_{m}}} & ( 9 )\end{matrix}$$

and Kᵤ is in the unfolding equilibrium constant for ERR-α dimers that is calculated from the melting curve of the protein in the absence of ligand as described by Pantoliano et al. (J. Biomolecular Screening, 6, 429, 2001) and Bowie & Sauer (Biochemistry, 28, 7139, 1989).

To compare dissociation constants at a common reference temperature, T_{ref}, the following equation was used:

$${K}_{d 1}^{{T}_{m}} = exp [ \frac{ln {K}_{d}^{ref} - Δ {H}_{b}^{ref} \times ( {T}_{m} - {T}_{ref} )}{R \times {T}_{ref} \times {T}_{m}} ] \qquad ( 10 )$$

where
K_{d}^{ref}=is the dissociation constant of the ligand at a reference temperature T_{ref}
ΔH_{b}^{ref}=is the binding enthalpy of the ligand to the protein at a reference temperature T_{ref}.

To solve for K_{d1}^{T}^{ₘ} from experiments and calculate K_{d}^{ref}, the following input parameters were used:
ΔHᵤ^{o}=165 kcal/mol and is unfolding enthalpy of the protein at T=Tₘ^{o} determined by the melting curve of the protein in the absence of ligand
Tₘ^{o}=325.25 K is the melting temperature of the protein in the absence of ligand
ΔCₚ=5 kcal/mol-K is the change in heat capacity for the unfolding of the protein in the absence of the ligand
Pₜ=4 μM is the total protein concentration determined by experimental design
Lₜ=100 μM is the total ligand concentration determined by experimental design
ΔH_{b}^{ref}=−5 kcal/mol is based on reasonable estimates from literature

In the thermodynamic treatment of the data the following assumptions were made: i) the small ligand interacts only with the folded state of the protein, ii) the reactions are reversible; iii) the unfolding protein reaction is a two-state process and iv) ideal dilute solutions are being used (specific activity for protein and ligands is equal to 1). All fitting and numerical integrations were done using the commercial program MicroMath® Scientist® version 2.01. The results are shown in Table 1 below.

A cell based reporter assay was also used to determine the functional response of the ERR-α hits. Transfections were performed in HEK293E cells that were maintained in DMEM supplemented in glutamine and 10% FBS. Co-transfections of 4 μg of a luciferase reporter plasmid and 4 μg of each pBIND-Gal4-ERR-α and pACT-SRC2 plasmids per T-75 flask were done using Lipofectamine as per manufacturers instructions. Twenty-four hours post-transfection, the cells were seeded in 96-well plates at density of 50,000 cells per well in assay media (DMEM phenol free, 5% charcoal stripped FBS). The cells were allowed to adhere to the bottom of the wells (approximately 5 hours post-seeding) and the compounds were dosed and the final concentration of DMSO was kept below 0.3%. After 24 hours of compound treatment cells were lysed and treated with the Promega Dual-Glo system. Firefly Luciferase activity was read using a luminescence plate reader, and data were normalized against Renilla luciferase activity. Data were fitted using subroutines available from GraphPad. The reported IC₅₀ values shown in the table below are the average from three independent experiments for the compounds tested.

TABLE 1
K_{d} and IC₅₀ from 2 Hybrid Assay Results

| | | | IC₅₀ 2-Hybrid cell based |
|---|---|---|---|
| Ex. # | TDP # | TF Kd (M) | assay (M) |
| 1 | 312569 | 6.07 × 10⁻⁶ | 6.07 × 10⁻⁶ |
| 2 | 492439 | 7.78 × 10⁻⁶ | 7.78 × 10⁻⁶ |
| 3 | 492531 | 1.71 × 10⁻⁶ | 1.71 × 10⁻⁶ |
| 4 | 514896 | 1.43 × 10⁻⁶ | 1.43 × 10⁻⁶ |
| 5 | 504540 | 2.6 × 10⁻⁵ | 2.6 × 10⁻⁵ |
| 6 | 504539 | N.D. | N.D. |
| 7 | 312570 | 3.59 × 10⁻⁷ | 0.5 × 10⁻⁶ |
| 8 | 514897 | 4.73 × 10⁻⁷ | 0.14 × 10⁻⁶ |
| 9 | 535754 | 2.47 × 10⁻⁵ | N.D. |
| 10 | 504542 | 2.34 × 10⁻⁷ | N.D. |
| 11 | 514918 | 5.29 × 10⁻⁷ | 1.0 × 10⁻⁶ |
| 12 | 514919 | 4.63 × 10⁻⁷ | 0.05 × 10⁻⁶ |
| 13 | 521424 | 7.95 × 10⁻⁵ | N.D. |
| 14 | 525345 | 5.88 × 10⁻⁷ | N.D. |
| 15 | 528344 | 1.14 × 10⁻⁷ | 1.5 × 10⁻⁶ |
| 16 | 528412 | 3.53 × 10⁻⁶ | N.D. |
| 17 | 536422 | 4.86 × 10⁻⁶ | N.D. |
| 18 | 536421 | 6.92 × 10⁻⁸ | N.D. |
| 19 | 545562 | 2.7 × 10⁻⁶ | N.D. |
| 20 | 514916 | 9.5 × 10⁻⁶ | N.D. |
| 21 | 521430 | >200 × 10⁻⁶ | N.D. |
| 22 | 504485 | 5.06 × 10⁻⁶ | N.D. |
| 23 | 525344 | 1.46 × 10⁻⁵ | N.D. |
| 24 | 504486 | 2.54 × 10⁻⁶ | N.D. |
| 25 | 527774 | 7.17 × 10⁻⁶ | N.D. |
| 26 | 312562 | 1.52 × 10⁻⁵ | N.D. |
| 27 | 527858 | 1.38 × 10⁻⁶ | N.D. |
| 28 | 535756 | 6.08 × 10⁻⁷ | N.D. |
| 29 | 535757 | 1.54 × 10⁻⁵ | N.D. |
| 30 | 525324 | 1.16 × 10⁻⁶ | N.D. |
| 31 | 525348 | 2.2 × 10⁻⁶ | N.D. |
| 32 | 535759 | 7.95 × 10⁻⁷ | N.D. |
| 33 | 535761 | 4.73 × 10⁻⁷ | N.D. |
| 34 | 535760 | N.D. | N.D. |
| 35 | 536419 | 5.2 × 10⁻⁷ | N.D. |
| 36 | 525347 | 2.03 × 10⁻⁵ | N.D. |
| 37 | 545567 | 4.14 × 10⁻⁷ | N.D. |
| 38 | 545563 | 3.18 × 10⁻⁶ | N.D. |
| 39 | 499127 | N.D. | N.D. |
| 40 | 384762 | 1.77 × 10⁻⁵ | N.D. |

N.D. = not determined

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A method of treating a subject suffering from or diagnosed with a disease, disorder, or medical condition mediated by ERR-α activity, comprising administering to the subject an effective amount to treat the disease, disorder, or medical condition of a compound of formula (I): 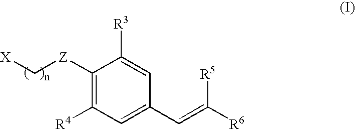
wherein:
n is 0 or 1;
Z is —O—, —S—, >NH, or >NR^{a} where R^{a} is alkyl, cycloalkyl, phenyl, or heterocycloalkyl;
X is an aryl or heteroaryl group;
R³ is —H or —O-alkyl unsubstituted or substituted with one or more substituents independently selected from the group consisting of —OH, halo, —CN, —O-alkyl, and —N(R^{w})R^{x} where R^{w} and R^{x} are each independently —H or alkyl;
R⁴ is selected from the group consisting of —H, halo, —O-alkyl, —CN, —NO₂, and —COOH; and
R⁵ and R⁶ are each independently —CN; —COOH; or a moiety selected from the group consisting of —COO-alkyl, —(C═O)alkyl, —(S═(O)ₘ)-aryl where m is 0, 1, or 2, cycloalkyl, heterocycloalkyl, —(C═O)phenyl, heteroaryl, and —(C═O)heterocycloalkyl; or R⁵ and R⁶ taken together with the carbon to which they are attached form an optionally benzofused heterocycloalkyl or cycloalkyl moiety; wherein each said moiety is unsubstituted or substituted with one or more substituents independently selected from the group consisting of: —OH; ═O; ═S; alkyl optionally substituted with —OH, —O-alkyl, phenyl, —NH₂, —NH(alkyl), —N(alkyl)₂, halo, —CF₃, —COOH, or —COO-alkyl; —O-alkyl; phenyl; —O-phenyl; benzyl; —O-benzyl; cycloalkyl; —O-cycloalkyl; —CN; —NO₂; —N(R^{y})R^{z} where R^{y} and R^{z} are each independently —H, alkyl, or —(C═O)alkyl, or R^{y} and R^{z} taken together with the nitrogen to which they are attached form a heterocycloalkyl wherein one carbon ring atom is optionally replaced with >O, >NH or >N-alkyl and where one carbon ring atom is optionally substituted with —OH or ═O; —(C═O)N(R^{y})R^{z}; —(N—R^{t})SO₂alkyl where R^{t} is —H or alkyl; —(C═O)alkyl; —(S═(O)ₙ)alkyl where n is 0, 1 or 2; —SO₂N(R^{y})R^{z} where R^{y} and R^{z} are as defined above; —SCF₃; halo; —CF₃; —OCF₃; —COOH; and —COOalkyl;
or a pharmaceutically acceptable salt, pharmaceutically acceptable prodrug, or pharmaceutically active metabolite thereof.

2. A method as defined in claim 1, wherein X is an aryl or heteroaryl group having one ring or two fused rings, wherein each ring has five or six ring atoms.

3. A method as defined in claim 2, wherein: Z is —O—; and
X is 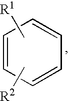
where
R¹ and R² are each independently —H; halo; —CN; —CF₃; —NO₂; —COOH; or a moiety selected from the group consisting of: —C₁₋₆alkyl, —OC₁₋₆alkyl, —C₂₋₆alkenyl, —OC₃₋₆alkenyl, —C₂₋₆alkynyl, —OC₃₋₆alkynyl, —C₃₋₇cycloalkyl, —(C₃₋₈cycloalkyl)C₁₋₆alkyl, —(C₃₋₈cycloalkyl)C₃₋₈alkenyl, —C₀₋₈alkylC(═O)C₁₋₈alkyl, 5-9 membered heterocycloalkyl, phenyl, —O-phenyl, benzyl, -(5-9-membered heterocycloalkyl)C₁₋₆alkyl, -(phenyl)C₁₋₆alkyl, —COOC₁₋₆alkyl, and —(C═O)N(R^{s})R^{t} where R^{s} and R^{t} are each independently —H or —C₁₋₆alkyl; wherein each said moiety is unsubstituted or substituted with one or more substituents independently selected from the group consisting of —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, and —N(R^{w})R^{x} where R^{w} and R^{x} are each independently —H or —C₁₋₆alkyl;
R⁴ is selected from the group consisting of —H, halo, —OC₁₋₆alkyl, —CN, —NO₂, and —COOH; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, and —(C═O)(5-9 membered heterocycloalkyl); or R⁵ and R⁶ taken together with the carbon to which they are attached form an optionally benzofused 5-9 membered heterocycloalkyl or cycloalkyl moiety; wherein each said moiety is unsubstituted or substituted with one or more substituents independently selected from the group consisting of: —OH; ═O; ═S; —C₁₋₆alkyl optionally substituted with —OH, —OC₁₋₆alkyl, phenyl, —NH₂, —NH(C₁₋₆alkyl), —N(C₁₋₆alkyl)₂, halo, —CF₃, —COOH, or —COOC₁₋₆alkyl; —OC₁₋₆alkyl; phenyl; —Ophenyl; benzyl; —Obenzyl; —C₃₋₆cycloalkyl; —OC₃₋₆cycloalkyl; —CN; —NO₂; —N(R^{y})R^{z} where R^{y} and R^{z} are each independently —H, —C₁₋₆alkyl, or —(C═O)C₁₋₆alkyl, or R^{y} and R^{z} taken together with the nitrogen to which they are attached form a 4-7 membered heterocycloalkyl ring wherein one carbon ring atom is optionally replaced with >O, >NH or >N(C₁₋₄alkyl) and where one carbon ring atom is optionally substituted with —OH or ═O; —(C═O)N(R^{y})R^{z}; —(N—R^{t})SO₂C₁₋₆alkyl where R^{t} is —H or —C₁₋₆alkyl; —(C═O)C₁₋₆alkyl; —(S═(O)ₙ)—C₁₋₆alkyl where n is 0, 1 or 2; —SO₂N(R^{y})R^{z} where R^{y} and R^{z} are as defined above; —SCF₃; halo; —CF₃; —OCF₃; —COOH; and —COOC₁₋₆alkyl.

4. A method according to claim 1, wherein the disease, disorder, or medical condition is selected from the group consisting of: bone-related disease, bone formation, cartilage formation, cartilage loss, cartilage degeneration, cartilage injury, ankylosing spondylitis, chronic back injury, gout, osteoporosis, osteolytic bone metastasis, multiple myeloma, chondrosarcoma, chondrodysplasia, osteogenesis imperfecta, osteomalacia, Paget's disease, polymyalgia rheumatica, pseudogout, arthritis, rheumatoid arthritis, infectious arthritis, osteoarthritis, psoriatic arthritis, reactive arthritis, childhood arthritis, Reiter's syndrome, and repetitive stress injury.

5. A method according to claim 1, wherein the disease, disorder, or medical condition is selected from the group consisting of: periodontal disease, chronic inflammatory airway disease, chronic bronchitis, and chronic obstructive pulmonary disease.

6. A method according to claim 1, wherein the disease, disorder, or medical condition is breast cancer.

7. A method according to claim 1, wherein the disease, disorder, or medical condition is selected from the group consisting of: metabolic syndrome, obesity, disorders of energy homeostasis, diabetes, lipid disorders, cardiovascular disorders, and artherosclerosis.

8. A method of treating a subject suffering from or diagnosed with a disease, disorder, or medical condition mediated by ERR-α activity, comprising administering to the subject a pharmaceutical composition comprising:
(a) an effective amount of a pharmaceutical agent to treat the disease, disorder, or medical condition, said pharmaceutical agent selected from the group consisting of compounds of formula (II): 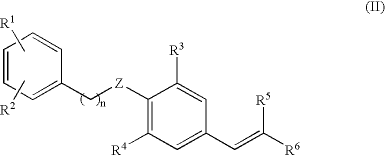
wherein
n is 0 or 1;
Z is —O—, —S—, >NH, or >NR^{a} where R^{a} is alkyl, —C₁₋₆cycloalkyl, phenyl, or 5-9-membered heterocycloalkyl;
R¹ and R² are each independently —H, halo, —CN, —CF₃, —NO₂, or —COOH, or a moiety selected from the group consisting of: —C₁₋₆alkyl, —OC₁₋₆alkyl, —C₂₋₆alkenyl, —OC₃₋₆alkenyl, —C₂₋₆alkynyl, —OC₃₋₆alkynyl, —C₃₋₇cycloalkyl, —(C₃₋₈cycloalkyl)C₁₋₆alkyl, —(C₃₋₈cycloalkyl)—C₃₋₈alkenyl, —C₀₋₈alkylC(═O)C₁₋₈alkyl, 5-9 membered heterocycloalkyl, phenyl, —O-phenyl, benzyl, -(5-9-membered heterocycloalkyl)C₁₋₆alkylene, -(phenyl)C₁₋₆alkyl, —COOC₁₋₆alkyl, and —(C═O)N(R^{s})R^{t} where R^{s} and R^{t} are each independently —H or —C₁₋₆alkyl; wherein each said moiety is unsubstituted or substituted with one or more substituents independently selected from the group consisting of —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, and —NR^{w}R^{x} where R^{w} and R^{x} are each independently —H or —C₁₋₆alkyl;
R⁴ is —H, —OCH₃, or —Cl; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, and —(C═O)(5-9 membered heterocycloalkyl); or R⁵ and R⁶ taken together with the carbon to which they are attached form a 5-9 membered heterocycloalkyl or cycloalkyl moiety; wherein each said moiety is unsubstituted or substituted with one or more substituents independently selected from the group consisting of: —OH; —C₁₋₆alkyl; —OC₁₋₆alkyl; —Ophenyl; benzyl; —Obenzyl; —C₃₋₆cycloalkyl; —OC₃₋₆cycloalkyl; —CN; —NO₂; —N(R^{y})R^{z} where R^{y} and R^{z} are each independently —H, —C₁₋₆alkyl, —C₁₋₆alkenyl, or —(C═O)C₁₋₆alkyl, or R^{y} and R^{z} taken together with the nitrogen to which they are attached form a 4-7 membered heterocycloalkyl ring wherein one carbon ring atom is optionally replaced with >O, ═N—, >NH or >N(C₁₋₄alkyl) and where one carbon ring atom is optionally substituted with —OH or ═O; —(C═O)N(R^{y})R^{z}; —(N—R^{t})SO₂C₁₋₆alkyl where R^{t} is —H or —C₁₋₆alkyl; —(C═O)C₁₋₆alkyl; —S═(O)ₙ)—C₁₋₆alkyl where n is 0, 1 or 2; —SO₂N(R^{y})R^{z} where R^{y} and R^{z} are as defined above; —SCF₃; halo; —CF₃; —OCF₃; —COOH; and —COOC₁₋₆alkyl;
and pharmaceutically acceptable salts, pharmaceutically acceptable prodrugs, and pharmaceutically active metabolites of said compounds; and
(b) a pharmaceutically acceptable excipient.

9. A method according to claim 8, wherein the disease, disorder, or medical condition is bone-related disease, bone formation, cartilage formation, cartilage loss, cartilage degeneration, cartilage injury, ankylosing spondylitis, chronic back injury, gout, osteoporosis, osteolytic bone metastasis, multiple myeloma, chondrosarcoma, chondrodysplasia, osteogenesis imperfecta, osteomalacia, Paget's disease, polymyalgia rheumatica, pseudogout, arthritis, rheumatoid arthritis, infectious arthritis, osteoarthritis, psoriatic arthritis, reactive arthritis, childhood arthritis, Reiter's syndrome, repetitive stress injury, periodontal disease, chronic inflammatory airway disease, chronic bronchitis, chronic obstructive pulmonary disease, breast cancer, metabolic syndrome, obesity, energy disorder, homeostasis, diabetes, lipid disorder, cardiovascular disorder, or artherosclerosis.

10. A method according to claim 9, wherein the pharmaceutical agent is a compound of the Formula (II) or a pharmaceutically acceptable salt thereof, wherein:
n is 0 or 1;
Z is —O—;
R¹ and R² are each independently —H, -halo, —CN, —CF₃, —NO₂, or —COOH, or a moiety selected from the group consisting of: —C₁₋₆alkyl, —OC₁₋₆alkyl, —C₃₋₇cycloalkyl, —(C═O)C₁₋₆alkyl, —COOC₁₋₆alkyl, —(C═O)N(R^{s})R^{t} where R^{s} and R^{t} are each independently —H or —C₁₋₆alkyl, wherein each said moiety is unsubstituted or substituted with one or more substituents independently selected from the group consisting of: —OH, halo, —CN, —CF₃, —OCF₃, —NO₂, and —COOC₁₋₆alkyl;
R³ is —H or —OC₁₋₆alkyl unsubstituted or substituted with one or more substituents independently selected from the group consisting of —OH, halo, —CN, —OC₁₋₆alkyl, or —NR^{w}R^{x} where R^{w} and R^{x} are each independently —H or —C₁₋₆alkyl;
R⁴ is —H or —Cl; and
R⁵ and R⁶ are each independently —CN; —COOH; or a moiety selected from the group consisting of —COOC₁₋₆alkyl, —(C═O)C₁₋₆alkyl, —(S═(O)ₘ)-aryl where m is 0, 1, or 2, —C₃₋₇cycloalkyl, 5-9 membered heterocycloalkyl, —(C═O)phenyl, heteroaryl, —(C═O)(5-9 membered heterocycloalkyl); or R⁵ and R⁶ taken together with the carbon to which they are attached form a 5-9 membered heterocycloalkyl or cycloalkyl moiety selected from the group consisting of: 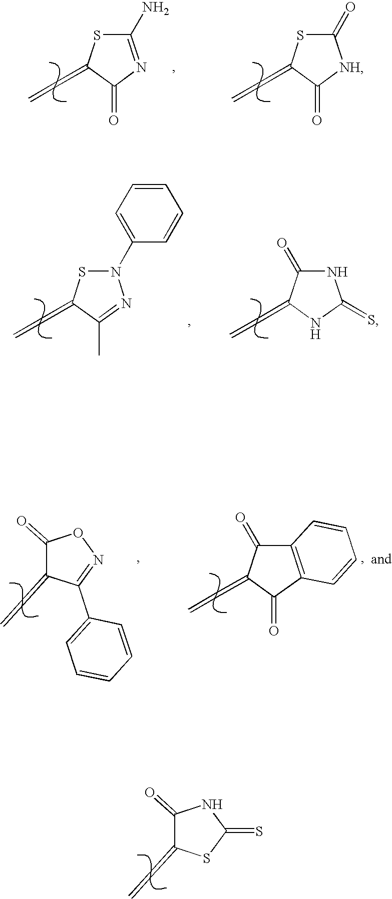
wherein each said moiety is unsubstituted or substituted with one or more substituents independently selected from the group consisting of: —OH; —C₁₋₄alkyl; —OC₁₋₃alkyl; phenyl; benzyl; —C₃₋₆cycloalkyl; —OC₃₋₆cycloalkyl; —CN; —NO₂; —N(R^{y})R^{z} where R^{y} and R^{z} are each independently —H or —C₁₋₆alkyl, or where R^{y} and R^{z} may be taken together with the nitrogen to which they are attached to form a 4-7 membered heterocycloalkyl ring wherein one carbon ring atom is optionally replaced with >O, ═N—, >NH or >N(C₁₋₄alkyl) and where one carbon ring atom is optionally substituted with —OH; —(C═O)N(R^{y})R^{z}; —(N—R^{t})SO₂C₁₋₆alkyl where R^{t} is —H or —C₁₋₆alkyl; —(C═O)C₁₋₆alkyl; —(S═(O)ₙ)—C₁₋₆alkyl where n is 0, 1 or 2; —SO₂N(R^{y})R^{z}; -halo; —CF₃; —OCF₃; —COOH; and —COOC₁₋₆alkyl.

11. A method according to claim 10, wherein:
n is 0 or 1;
Z is —O—;
R¹ and R² are each independently selected from the group consisting of: —H, —OCH₃, —F, —Cl, —CN, —CF₃, —NO₂, and —COOCH₃;
R³ is —H or —OCH₃;
R⁴ is —H or —Cl; and
R⁵ and R⁶ are each independently —CN; or a moiety selected from the group consisting of —COOC₁₋₆alkyl, —(C═O)phenyl, and 3-pyrazolyl; or R⁵ and R⁶ taken together with the carbon to which they are attached form a 5-9 membered heterocyclic or carbocyclic moiety selected from the group consisting of: 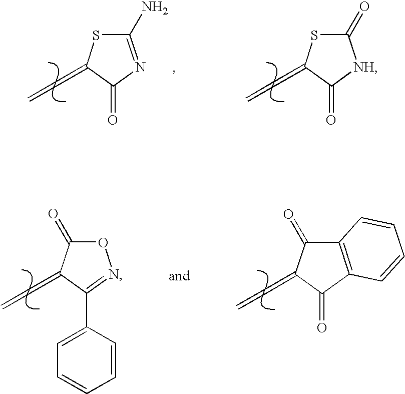
each unsubstituted or substituted with one or more substituents selected from the group consisting of: —OH, —C₁₋₄alkyl, —OC₁₋₃alkyl, phenyl, —C₃₋₆cycloalkyl, —OC₃₋₆cycloalkyl, —CN, —NO₂, —NH₂, —N(C₁₋₃alkyl)₂, —-N-piperidinyl, —N-morpholinyl, —N-thiomorpholinyl, —(C═O)N(C₁₋₃alkyl)₂, —(N—R^{t})SO₂C₁₋₃alkyl where R^{t} is —H or —C₁₋₆alkyl, —(C═O)C₁₋₃alkyl, —(S═(O)ₙ)—C₁₋₃alkyl where n is 0, 1 or 2, —SO₂N(C₁₋₃alkyl)₂, -halo, —CF₃, —OCF₃, —COOH, and —COOC₁₋₆alkyl.

12. A method according to claim 8, wherein said compound is selected from the group consisting of:
5-Amino-3-{1-cyano-2-[3-methoxy-4-(2-nitro-4-trifluoromethyl-phenoxy)-phenyl]vinyl}-1-phenyl-1H-pyrazole-4-carbonitrile;
5-Amino-3-{1-cyano-2-[4-(2-nitro-4-trifluoromethyl-phenoxy)-phenyl]-vinyl}-1-phenyl-1H-pyrazole-4-carbonitrile;
5-Amino-3-{1-cyano-2-[3-methoxy-4-(4-nitro-3-trifluoromethyl-phenoxy)-phenyl]-vinyl}-1-phenyl-1H-pyrazole-4-carbonitrile;
5-Amino-3-{1-cyano-2-[4-(4-cyano-3-trifluoromethyl-phenoxy)-3-methoxy-phenyl]-vinyl}-1-phenyl-1H-pyrazole-4-carbonitrile;
5-Amino-3-[1-cyano-2-(4-phenoxy-phenyl)-vinyl]-1-phenyl-1H-pyrazole-4-carbonitrile;
5-Amino-3-[2-(4-benzyloxy-3-methoxy-phenyl)-1-cyano-vinyl]-1-phenyl-1H-pyrazole-4-carbonitrile;
2-Amino-5-[3-methoxy-4-(2-nitro-4-trifluoromethyl-phenoxy)-benzylidene]-thiazol-4-one;
4-[4-(2-Amino-4-oxo-4H-thiazol-5-ylidenemethyl)-2-methoxy-phenoxy]-2-trifluoromethyl-benzonitrile;
2-Amino-5-[4-(4-methoxy-phenoxy)-benzylidene]-thiazol-4-one;
5-[3-Methoxy-4-(2-nitro-4-trifluoromethyl-phenoxy)-benzylidene]-thiazolidine-2,4-dione;
5-[3-Methoxy-4-(4-nitro-3-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzonitrile;
5-[3-Chloro-5-methoxy-4-(2-nitro-4-trifluoromethyl-phenoxy)-benzylidene]-thiazolidine-2,4-dione;
5-[4-(2-Nitro-4-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzoic acid methyl ester;
5-[4-(4-Methoxy-phenoxy)-benzylidene]-thiazolidine-2,4-dione;
{5-[3-Methoxy-4-(2-nitro-4-trifluoromethyl-phenoxy)-benzylidene]-2,4-dioxo-thiazolidin-3-yl}-acetic acid ethyl ester;
4-[4-(2,4-Dioxo-thiazolidin-5-ylidenemethyl)-2-methoxy-phenoxy]-3-nitro-benzoic acid methyl ester;
3-Butyl-5-[3-methoxy-4-(2-nitro-4-trifluoromethyl-phenoxy)-benzylidene]-thiazolidine-2,4-dione;
5-(4-Phenoxy-benzylidene)-thiazolidine-2,4-dione;
5-[3-(3-Chloro-phenoxy)-benzylidene]-thiazolidine-2,4-dione;
2-[3-Methoxy-4-(2-nitro-4-trifluoromethyl-phenoxy)-benzylidene]-malononitrile;
2-Benzenesulfonyl-3-[3-methoxy-4-(2-nitro-4-trifluoromethyl-phenoxy)-phenyl]-acrylonitrile;
2-Cyano-3-[3-methoxy-4-(2-nitro-4-trifluoromethyl-phenoxy)-phenyl]-acrylic acid ethyl ester;
3-[3-Methoxy-4-(2-nitro-4-trifluoromethyl-phenoxy)-phenyl]-2-thiophen-2-yl-acrylonitrile;
2-(1H-Benzoimidazol-2-yl)-3-[3-methoxy-4-(2-nitro-4-trifluoromethyl-phenoxy)-phenyl]-acrylonitrile;
3-[3-Methoxy-4-(2-nitro-4-trifluoromethyl-phenoxy)-phenyl]-2-pyridin-2-yl-acrylonitrile;
2-Benzoyl-3-[3-methoxy-4-(2-nitro-4-trifluoromethyl-phenoxy)-phenyl]-acrylonitrile;
4,4,4-Trifluoro-2-[3-methoxy-4-(2-nitro-4-trifluoromethyl-phenoxy)-benzylidene]-1-thiophen-2-yl-butane-1,3-dione;
4-[3-Methoxy-4-(2-nitro-4-trifluoromethyl-phenoxy)-benzylidene]-5-methyl-2-phenyl-2,4-dihydro-pyrazol-3-one;
5-[3-Methoxy-4-(2-nitro-4-trifluoromethyl-phenoxy)-benzylidene]-2-thioxo-imidazolidin-4-one;
4-[3-Methoxy-4-(2-nitro-4-trifluoromethyl-phenoxy)-benzylidene]-3-phenyl-4H-isoxazol-5-one;
2-[3-Methoxy-4-(2-nitro-4-trifluoromethyl-phenoxy)-benzylidene]-indan-1,3-dione.
5-[3-Methoxy-4-(2-nitro-4-trifluoromethyl-phenoxy)-benzylidene]-3-phenyl-2-thioxo-imidazolidin-4-one;
4-[4-(1,3-Dioxo-indan-2-ylidenemethyl)-2-methoxy-phenoxy]-3-nitro-benzoic acid methyl ester;
3-Ethyl-5-[3-methoxy-4-(2-nitro-4-trifluoromethyl-phenoxy)-benzylidene]-2-thioxo-thiazolidin-4-one;
5-[4-(2-Chloro-4-fluoro-benzyloxy)-3-methoxy-benzylidene]-thiazolidine-2,4-dione;
5-[4-(3-Fluoro-benzyloxy)-3-methoxy-benzylidene]-thiazolidine-2,4-dione;
5-(4-Benzyloxy-3-methoxy-benzylidene)-thiazolidine-2,4-dione; and
2-[4-(3-Fluoro-benzyloxy)-3-methoxy-benzylidene]-indan-1,3-dione.

13. A method according to claim 2 wherein said compound is selected from the group consisting of:
5-Amino-3-{1-cyano-2-[3-methoxy-4-(4-nitro-3-trifluoromethyl-phenoxy)-phenyl]-vinyl}-1-phenyl-1H-pyrazole-4-carbonitrile;
5-Amino-3-{1-cyano-2-[4-(4-cyano-3-trifluoromethyl-phenoxy)-3-methoxy-phenyl]-vinyl}-1-phenyl-1H-pyrazole-4-carbonitrile;
2-Amino-5-[3-methoxy-4-(2-nitro-4-trifluoromethyl-phenoxy)-benzylidene]-thiazol-4-one;
4-[4-(2-Amino-4-oxo-4H-thiazol-5-ylidenemethyl)-2-methoxy-phenoxy]-2-trifluoromethyl-benzonitrile;
5-[3-Methoxy-4-(2-nitro-4-trifluoromethyl-phenoxy)-benzylidene]-thiazolidine-2,4-dione;
5-[3-Methoxy-4-(4-nitro-3-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzonitrile;
5-[4-(2-Nitro-4-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzoic acid methyl ester;
4-[4-(2,4-Dioxo-thiazolidin-5-ylidenemethyl)-2-methoxy-phenoxy]-3-nitro-benzoic acid methyl ester;
3-[3-Methoxy-4-(2-nitro-4-trifluoromethyl-phenoxy)-phenyl]-2-pyridin-2-yl-acrylonitrile;
2-Benzoyl-3-[3-methoxy-4-(2-nitro-4-trifluoromethyl-phenoxy)-phenyl]-acrylonitrile;
4-[3-Methoxy-4-(2-nitro-4-trifluoromethyl-phenoxy)-benzylidene]-5-methyl-2-phenyl-2,4-dihydro-pyrazol-3-one;
5-[3-Methoxy-4-(2-nitro-4-trifluoromethyl-phenoxy)-benzylidene]-2-thioxo-imidazolidin-4-one;
4-[3-Methoxy-4-(2-nitro-4-trifluoromethyl-phenoxy)-benzylidene]-3-phenyl-4H-isoxazol-5-one;
2-[3-Methoxy-4-(2-nitro-4-trifluoromethyl-phenoxy)-benzylidene]-indan-1,3-dione.
4-[4-(1,3-Dioxo-indan-2-ylidenemethyl)-2-methoxy-phenoxy]-3-nitro-benzoic acid methyl ester; and
5-[4-(2-Chloro-4-fluoro-benzyloxy)-3-methoxy-benzylidene]-thiazolidine-2,4-dione.

14. A method according to claim 2 wherein said compound is selected from the group consisting of:
2-Amino-5-[3-methoxy-4-(2-nitro-4-trifluoromethyl-phenoxy)-benzylidene]-thiazol-4-one; p1 4-[4-(2-Amino-4-oxo-4H-thiazol-5-ylidenemethyl)-2-methoxy-phenoxy]-2-trifluoromethyl-benzonitrile;
5-[3-Methoxy-4-(2-nitro-4-trifluoromethyl-phenoxy)-benzylidene]-thiazolidine-2,4-dione;
5-[3-Methoxy-4-(4-nitro-3-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzonitrile;
5-[4-(2-Nitro-4-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzoic acid methyl ester;
4-[4-(2,4-Dioxo-thiazolidin-5-ylidenemethyl)-2-methoxy-phenoxy]-3-nitro-benzoic acid methyl ester;
2-Benzoyl-3-[3-methoxy-4-(2-nitro-4-trifluoromethyl-phenoxy)-phenyl]-acrylonitrile;
2-[3-Methoxy-4-(2-nitro-4-trifluoromethyl-phenoxy)-benzylidene]-indan-1,3-dione.
4-[4-(1,3-Dioxo-indan-2-ylidenemethyl)-2-methoxy-phenoxy]-3-nitro-benzoic acid methyl ester; and
5-[4-(2-Chloro-4-fluoro-benzyloxy)-3-methoxy-benzylidene]-thiazolidine-2,4-dione.

15. A method according to claim 2 wherein said compound is selected from the group consisting of:
5-[3-Methoxy-4-(2-nitro-4-trifluoromethyl-phenoxy)-benzylidene]-thiazolidine-2,4-dione;
4-[4-(2,4-Dioxo-thiazolidin-5-ylidenemethyl)-2-methoxy-phenoxy]-2-trifluoromethyl-benzoic acid methyl ester;
4-[4-(2,4-Dioxo-thiazolidin-5-ylidenemethyl)-2-methoxy-phenoxy]-3-nitro-benzoic acid methyl ester;
2-Benzoyl-3-[3-methoxy-4-(2-nitro-4-trifluoromethyl-phenoxy)-phenyl]-acrylonitrile; and
4-[3-Methoxy-4-(2-nitro-4-trifluoromethyl-phenoxy)-benzylidene]-5-methyl-2-phenyl-2,4-dihydro-pyrazol-3-one.
